(19) United States
(12) Patent Application Publication
(10) Pub. No.: US 2024/0358320 A1
(43) Pub. Date: Oct. 31, 2024

(54) DIABETES SENSOR, MANUFACTURING METHOD THEREOF, AND CLOSED-LOOP CONTROL SYSTEM

(71) Applicant: PEKING UNIVERSITY, Beijing (CN)

(72) Inventor: Yue CUI, Beijing (CN)

(21) Appl. No.: 18/644,402

(22) Filed: Apr. 24, 2024

(30) Foreign Application Priority Data
Apr. 27, 2023 (CN) 202310473415.X
Apr. 27, 2023 (CN) 202320993680.6
Apr. 27, 2023 (CN) 202320999725.0
Apr. 27, 2023 (CN) 202321002223.2

(51) Int. Cl. A61B 5/00; A61B 5/145; A61B 5/1486; A61M 37/00

(52) U.S. Cl. CPC A61B 5/4836; A61B 5/14532; A61B 5/1486; A61M 37/0015; A61M 2037/0023; A61M 2037/0053; A61M 2037/0061

(57) ABSTRACT

Disclosed are a diabetes sensor, a method for manufacturing the diabetes sensor, and a closed-loop control system. The diabetes sensor includes a substrate, a microneedle array arranged on one side of the substrate, and a plurality of electrodes covering the microneedle array and the substrate, wherein the microneedle array includes a plurality of microneedles; and the plurality of electrodes includes an electrochemical sensor and a reverse iontophoresis device; the electrochemical sensor being configured to detect glucose molecules in interstitial fluid and generate an electrical signal; and the reverse iontophoresis device being configured to generate a reverse iontophoresis effect to attract glucose molecules in a deep skin layer to an upper part of dermis where needle tips of the microneedles are located.

### CROSS REFERENCE TO RELATED APPLICATION

This patent application claims the benefits and priorities of the following four Chinese Patent Applications: Chinese Patent Application No. 202310473415.X, entitled “DIABETES SENSOR AND MANUFACTURING METHOD THEREOF”; Chinese Patent Application No. 2023209936806, entitled “CLOSED-LOOP CONTROL SYSTEM”; Chinese Patent Application No. 2023209997250, entitled “CLOSED-LOOP CONTROL SYSTEM”; and Chinese Patent Application No. 2023210022232, entitled “CLOSED-LOOP CONTROL SYSTEM”. All of the four Chinese Patent Applications are filed with the China National Intellectual Property Administration on Apr. 27, 2023. The disclosure of the four applications is incorporated by references herein in their entireties as part of the present application.

### TECHNICAL FIELD

The present disclosure relates to the technical field of medical auxiliary devices, in particular to a diabetes sensor, a manufacturing method thereof, and a closed-loop control system.

### BACKGROUND ART

Diabetes is a disease based on metabolic abnormalities, which is caused by insufficient insulin, a hormone secreted by the pancreas. As one of the active treatments, insulin could be injected by a patient with diabetes into his/her body. Insulin injection device could be used to appropriately inject insulin into the body according to the blood glucose changes of patients. However, the existing insulin injection devices generally have a needle tube structure, which brings a strong pain feeling during the injection and causes infection easily.

### SUMMARY

An object of the present disclosure is to provide a diabetes sensor, a method for manufacturing the diabetes sensor, and a closed-loop control system, to solve the problems exiting in the prior art, improve the comfort level when using microneedles and further reduce the risk of infection.

To achieve the above object, the present disclosure provides the following technical solutions.

The present disclosure provides a diabetes sensor, including:
a substrate, a microneedle array arranged on one side of the substrate, and a plurality of electrodes covering the microneedle array and the substrate,
wherein the microneedle array includes a plurality of microneedles; and
wherein the plurality of electrodes includes an electrochemical sensor and a reverse iontophoresis device,
the electrochemical sensor being configured to detect glucose molecules in interstitial fluid and generate an electric signal, and
the reverse iontophoresis device being configured to generate a reverse iontophoresis effect to attract the glucose molecules from a deep skin layer to an upper part of dermis where needle tips of the microneedle are located.

In some embodiments, the microneedles each have a height of not less than 100 μm and not more than 1000 μm.

In some embodiments, the electrochemical sensor includes a working electrode and a counter electrode, or includes a working electrode, a reference electrode, and a counter electrode; and the reverse iontophoresis device includes a positive electrode and a negative electrode; wherein the working electrode of the electrochemical sensor and the negative electrode of the reverse iontophoresis device form the interdigital electrodes; glucose oxidase is immobilized on the working electrode of the electrochemical sensor; and the counter electrode of the electrochemical sensor and the positive electrode of the reverse iontophoresis device are located on one side or two sides of the interdigital electrodes.

In some embodiments, the microneedle array includes a solid microneedle array or a hollow microneedle array.

In some embodiments, the substrate and the microneedle array are each independently made of a material including one selected from the group consisting of a polymeric material, a biodegradable material, and a biocompatible material.

In some embodiments, the working electrode of the electrochemical sensor is made of a material including one selected from the group consisting of gold, platinum, carbon, a gold composite, a platinum composite, a carbon composite, and silver/silver chloride; the counter electrode of the electrochemical sensor is made of a material including one selected from the group consisting of gold, platinum, carbon, a gold composite, a platinum composite, a carbon composite, and silver/silver chloride; and the reverse iontophoresis device is made of a material including one selected from the group consisting of silver/silver chloride, a silicone material, a conductive polymer, graphene, and gold.

The present disclosure provides a method for manufacturing a diabetes sensor, being applicable to the diabetes sensor as described above, including the steps of providing a substrate; forming a microneedle array on one side of the substrate, wherein the microneedle array includes a plurality of microneedles; and forming a plurality of electrodes on the substrate and the microneedle array, wherein the plurality of electrodes includes an electrochemical sensor and a reverse iontophoresis device, the electrochemical sensor being configured to detect glucose molecules in interstitial fluid and generate an electric signal; and the reverse iontophoresis device being configured to generate a reverse iontophoresis effect to attract the glucose molecules from a deep skin layer to an upper part of dermis where the needle tips of the microneedle is located.

In some embodiments, forming the microneedle array on one side of the substrate includes providing a mold with a microneedle sequence that matches the microneedle array; and pouring a polymeric material into the mold, solidifying the polymeric material, and then peeling a resulting solidified polymeric material off the mold, to obtain the microneedle array.

In some embodiments, forming the microneedle array on one side of the substrate includes forming the microneedle array on the substrate by a 3D printing process or a micro/nanofabrication process.

In some embodiments, forming the plurality of electrodes on the substrate and the microneedle array includes forming the plurality of electrodes by a micro/nanofabrication process, a screen printing process, or an aerosol jet printing process.

The present disclosure also provides a closed-loop control system, including a pump, a signal conversion module, and the diabetes sensor, wherein one end of the pump is connected with the substrate of the diabetes sensor, and a tip end of the microneedle array faces a side away from the pump; and the signal conversion module includes a first conversion module, a control module, and a second conversion module, wherein an input end of the first conversion module is connected with an output end of the diabetes sensor, and an output end of the first conversion module is connected with an input end of the control module, and the first conversion module is configured to receive and convert an electrical signal output by the diabetes sensor; the control module is configured to receive the electrical signal converted by the first conversion module and send an instruction to the second conversion module according to the electrical signal received; and an input end of the second conversion module is connected with the output end of the control module, and an output end of the second conversion module is connected with an input end of the pump; and the second conversion module is configured to receive and convert the instruction output by the control module, and send converted instruction signal to the pump to control opening or closing of the pump.

In some embodiments, the first conversion module is a first signal converter;

the control module is a microcontroller; and the second conversion module is a second signal converter.

In some embodiments, the pump is an ultrasonic pump, the ultrasonic pump including an upper casing, a lower casing, and a thin film arranged between the upper casing and the lower casing, wherein a drug storage chamber is formed between the thin film and the upper casing, a plurality of conical holes are distributed on the thin film, and a large-diameter end of each of the conical holes is adjacent to the drug storage chamber; a piezoelectric circular ring is arranged on one side of the thin film facing away from the drug storage chamber; the lower casing is provided with a liquid outlet, the liquid outlet being connected with the substrate of the diabetes sensor; and the tip end of the microneedle array faces a side away from the liquid outlet.

In some embodiments, the thin film is made of a material including at least one selected from the group consisting of stainless steel, gold, copper, zinc, platinum, silver, tungsten, aluminum, an aluminum alloy, natural rubber, isoprene rubber, polybutadiene rubber, styrene butadiene rubber, nitrile rubber, chloroprene rubber, butyl rubber, halogenated butyl rubber, ethylene propylene rubber, ethylene propylene diene rubber, epichlorohydrin rubber, polyacrylate rubber, silicone rubber, fluorosilicone rubber, fluororubber, chlorosulfonated polyethylene, hydrogenated nitrile rubber, thermoplastic polyolefin elastomer, thermoplastic styrene elastomer, polyurethane thermoplastic elastomer, polyester thermoplastic elastomer, polyamide thermoplastic elastomer, halogen containing thermoplastic elastomer, ionic thermoplastic elastomer, ethylene copolymer thermoplastic elastomer, 1,2-poly-butadiene thermoplastic elastomer, trans polyisoprene thermoplastic elastomer, melt processible thermoplastic elastomer, thermoplastic vulcanizate, and polydimethylsiloxane; and the piezoelectric ring is made of a piezoelectric crystal, a piezoelectric ceramic, and a piezoelectric polymer.

In some embodiments, the pump is an electroosmotic pump, the electroosmotic pump including a first electrode layer, a second electrode layer, and an intermediate film layer, wherein the intermediate film layer is located between the first electrode layer and the second electrode layer, and a plurality of perforations are distributed on the intermediate film layer; and the substrate is connected with the second electrode layer, and the tip end of the microneedle array faces a side away from the second electrode layer.

In some embodiments, the first electrode layer, the second electrode layer, and the intermediate film layer are each independently made of a material including a hard film material or a flexible film material.

In some embodiments, the pump is an electrochemical pump, the electrochemical pump including a pump body, wherein the pump body has an accommodation zone accommodating an electrolyte solution and an electrode layer connected to an inner wall of the pump body; the pump body is provided with an expanded film covering the accommodation zone; the expanded film is connected with the substrate of the diabetes sensor, and the tip end of the microneedle array faces a side away from the expanded film.

In some embodiments, the expanded film is made of a material comprising at least one selected from the group consisting of polytetrafluoroethylene, polydimethylsiloxane, polyacrylate, silicone, rubber, latex, polyurethane, parylene, and polyimide; and the electrode layer is made of a material comprising a hard film material or a flexible film material.

Embodiments of the present disclosure has the following technical effects compared with the prior art:

The present disclosure provides a diabetes sensor and a manufacturing method thereof. The diabetes sensor comprises a substrate, hollow microneedle array and a plurality of electrodes. The plurality of electrodes includes an electrochemical sensor and a reverse iontophoresis device. The electrochemical sensor is configured to detect glucose molecules in interstitial fluid and generate an electrical signal. The reverse iontophoresis device is configured to generate the reverse iontophoresis effect to attract the glucose molecules in the deep skin layer to the upper part of the dermis where the needle tips of the microneedles are located. When the diabetes sensor works, a constant current power supply provides power to the reverse iontophoresis device to attract subcutaneous glucose to the position of the negative electrode of the reverse iontophoresis device and the working electrode of the electrochemical sensor for detection. Further, the intensity of reverse iontophoresis is further enhanced as the microneedles penetrate the high-resistance epidermal layer, which attracts more glucose molecules to reach the vicinity of the electrode, thereby improving the accuracy and reliability of the diabetes sensor. The microneedles having such height do not reach the nerve layer, which reduces pain and infection risk, thereby improving the comfort level when using microneedles.

The closed-loop control system according to the present disclosure is provided with a pump, a diabetes sensor, and a control module, wherein the pump is connected with the substrate of the diabetes sensor, the input end of the control module is connected with the output end of the diabetes sensor, and the output end of the control module is connected with the input end of the pump. The glucose concentration in subcutaneous interstitial fluid of patients could be measured by using the diabetes sensor. Because the glucose concentration in interstitial fluid is highly correlated with the blood glucose concentration, the signal output by the diabetes sensor could reflect the blood glucose concentration. Also, the diabetes sensor could output signal(s) to the control module, and the control module could then control opening or closing of the pump according to electrical signal(s) output by the diabetes sensor, such that the diabetes sensor could inject insulin according to the blood glucose concentration of the patient in real time.

### BRIEF DESCRIPTION OF THE DRAWINGS

In order to more clearly illustrate the embodiments of the present disclosure or the technical solutions in the prior art, the following will briefly introduce the drawings needed to be used in the embodiments. It is obvious that the drawings described below are only some embodiments of the disclosure. For persons of ordinary skill in the art, other drawings could be obtained according to these drawings without creative labor.

FIG. 1 shows a schematic diagram of the structure of a diabetes sensor according to an embodiment of the disclosure.

FIG. 2 shows a schematic diagram of the principle of a diabetes sensor according to an embodiment of the disclosure.

FIG. 3 shows a schematic diagram of the sectional structure of a hollow microneedle array included in a diabetes sensor according to an embodiment of the disclosure.

FIG. 4 shows a schematic diagram of the sectional structure of a solid microneedle array included in a diabetes sensor according to an embodiment of the disclosure.

FIG. 5 shows a schematic diagram of the structures of an electrochemical sensor and a reverse iontophoresis device in a diabetes sensor according to an embodiment of the disclosure.

FIG. 6 shows a schematic diagram of the structure of a diabetes sensor according to an embodiment of the disclosure, in which the counter electrode of the electrochemical sensor and the positive electrode of the reverse iontophoresis device are located on the left side of the interdigital electrodes.

FIG. 7 shows a schematic diagram of the structure of a diabetes sensor according to an embodiment of the disclosure, in which the counter electrode of the electrochemical sensor and the positive electrode of the reverse iontophoresis device are located on the right side of the interdigital electrodes.

FIG. 8 shows a schematic diagram of the structure of an electrochemical sensor and a reverse iontophoresis device according to an embodiment of the disclosure, in which the electrochemical sensor includes a working electrode, a counter electrode, and a reference electrode.

FIG. 9 shows a schematic diagram of the structure of a diabetes sensor with pyramid microneedles according to an embodiment of the disclosure.

FIG. 10 shows a schematic diagram of the structure of a diabetes sensor including a working electrode, a reference electrode, and a counter electrode according to an embodiment of the disclosure.

FIG. 11 shows a flowchart of a method for manufacturing a diabetes sensor according to an embodiment of the disclosure.

FIG. 12 shows a flowchart of manufacturing microneedle array by soft lithography and manufacturing electrodes by micro/nanofabrication in the method for manufacturing a diabetes sensor according to an embodiment of the disclosure.

FIG. 13 shows a flowchart of manufacturing microneedle array by soft lithography and manufacturing electrodes by a screen printing process in the method for manufacturing a diabetes sensor according to an embodiment of the disclosure.

FIG. 14 shows a flowchart of manufacturing microneedle array by soft lithography and manufacturing electrodes by an aerosol jet printing process in the method for manufacturing a diabetes sensor according to an embodiment of the disclosure.

FIG. 15 shows a flowchart of manufacturing microneedle array by a 3D printing process and manufacturing electrodes by a micro/nanofabrication process in the method for manufacturing a diabetes sensor according to an embodiment of the disclosure.

FIG. 16 shows a flowchart of manufacturing microneedle array by a 3D printing process and manufacturing electrodes by a screen printing process in the method for manufacturing a diabetes sensor according to an embodiment of the disclosure.

FIG. 17 shows a flowchart of manufacturing microneedle array by a 3D printing process and manufacturing electrodes by an aerosol jet printing process in the method for manufacturing a diabetes sensor according to an embodiment of the disclosure.

FIG. 18 shows a flowchart of manufacturing microneedle array and electrodes by a micro/nanofabrication process in the method for manufacturing a diabetes sensor according to an embodiment of the disclosure.

FIG. 19 shows a flowchart of manufacturing microneedle array by a micro/nanofabrication process and manufacturing electrodes by a screen printing process in the method for manufacturing a diabetes sensor according to an embodiment of the disclosure.

FIG. 20 shows a flowchart of manufacturing microneedle array by a micro/nanofabrication process and manufacturing electrode by an aerosol jet printing process in the method for manufacturing a diabetes sensor according to an embodiment of the disclosure.

FIG. 21 is a curve chart showing current response of the diabetes sensor to different glucose concentrations, after application of a reverse iontophoresis current of 0.3 mA for 200 s according to an embodiment of the disclosure.

FIG. 22 shows a line chart of blood glucose concentration variations with current after application of a reverse iontophoretic current of 0.3 mA for 200 s according to an embodiment of the disclosure.

FIG. 23 is a curve chart showing a current response of the diabetes sensor to different concentrations of glucose without application of the reverse iontophoresis current according to an embodiment of the disclosure.

FIG. 24 shows a line chart of blood glucose concentration variations with current, without application of reverse iontophoresis current according to an embodiment of the disclosure.

FIG. 25 shows the trends of the current variations of the diabetes sensor and the blood glucose concentration variations over time after insulin injection without application of the reverse iontophoresis current according to an embodiment of the disclosure.

FIG. 26 shows a distribution diagram of predicted blood glucose concentrations after insulin injection without application of reverse iontophoretic current according to an embodiment of the disclosure.

FIG. 27 shows a histogram of error values of different blood glucose concentrations after insulin injection without application of reverse iontophoretic current according to an embodiment of the disclosure.

FIG. 28 shows the trends of the current variations of the diabetes sensor and the blood glucose concentration variations over time after insulin injection with application of the reverse iontophoresis current according to an embodiment of the disclosure.

FIG. 29 shows a distribution diagram of predicted blood glucose concentrations after insulin injection with application of reverse iontophoretic current according to an embodiment of the disclosure.

FIG. 30 shows a histogram of error values of different blood glucose concentrations after insulin injection with application of reverse iontophoretic current according to an embodiment of the disclosure.

FIG. 31 shows the trends of the current variations of the diabetes sensor and the blood glucose concentration variations after glucose injection with application of the reverse iontophoresis current according to an embodiment of the disclosure.

FIG. 32 shows a distribution diagram of predicted blood glucose concentrations after glucose injection with application of the reverse iontophoretic current according to an embodiment of the disclosure.

FIG. 33 shows a histogram of error values of different blood glucose concentrations after glucose injection with applying reverse iontophoretic current according to an embodiment of the disclosure.

FIG. 34 shows a comparison between the accurate values and the values measured by the diabetes sensor after application of the reverse iontophoresis current according to an embodiment of the disclosure.

FIG. 35 shows the prediction error percentages of different blood glucose concentrations before and after application of a reverse iontophoretic current according to an embodiment of the disclosure.

FIG. 36 shows prediction error percentages of blood glucose concentrations before and after application of a reverse iontophoretic current according to an embodiment of the disclosure.

FIG. 37 shows a structural diagram of a closed-loop control system according to an embodiment of the disclosure.

FIG. 38 shows a schematic diagram of the structure of an ultrasonic pump in the closed-loop control system according to an embodiment of the disclosure.

FIG. 39 shows a schematic diagram of the structure of a signal conversion module in the closed-loop control system according to an embodiment of the disclosure.

FIG. 40 shows a schematic diagram of the structure of the closed-loop control system according to an embodiment of the disclosure.

FIG. 41 shows a schematic diagram of the structure of an electroosmotic pump in the closed-loop control system according to an embodiment of the disclosure.

FIG. 42 shows a schematic diagram of the structure of a curved electroosmotic pump in the closed-loop control system according to an embodiment of the disclosure.

FIG. 43 shows a schematic diagram of the structure of a signal conversion module in the closed-loop control system according to an embodiment of the disclosure.

FIG. 44 shows a schematic diagram of the structure of the closed-loop control system according to an embodiment of the disclosure.

FIG. 45 shows a schematic diagram of the structure of an electrode layer of the electrochemical pump in the closed-loop control system according to an embodiment of the disclosure.

FIG. 46 shows a schematic diagram of the structure of an electrode layer in a sawtooth shape of the electrochemical pump in the closed-loop control system according to an embodiment of the disclosure.

FIG. 47 shows a schematic diagram of the structure of an electrode layer in a wave shape of an electrochemical pump in the closed-loop control system according to an embodiment of the disclosure.

FIG. 48 shows a schematic diagram of the structure of a signal conversion module in the closed-loop control system according to an embodiment of the disclosure.

Description of symbols in the figures: 1 represents a diabetes sensor; 10 represents a substrate; 11 represents a microneedle array; 111 represents microneedle(s); 12 represents electrode(s); 121 represents an electrochemical sensor; 1211 represents a working electrode; 1212 represents a counter electrode; 1213 represents a reference electrode; 122 represents a reverse iontophoresis device; 1221 represents a negative electrode; 1222 represents a positive electrode; 13 represents interdigital electrodes; 20 represents glucose molecules; 2 represents an ultrasonic pump; 21 represents an upper casing; 211 represents a liquid inlet; 212 represents a rubber plug; 22 represents a lower casing; 221 represents a liquid outlet; 23 represents a first sealing ring; 24 represents a thin film; 25 represents a piezoelectric ring; 26 represents a second sealing ring; 27 represents a drug storage chamber; 28 represents conical hole(s); 3 represents a signal conversion module; 31 represents a first conversion module; 32 represents a control module; 33 represents a second conversion module; 4 represents an electroosmotic pump; 41 represents a first electrode layer; 42 represents a second electrode layer; 43 represents an intermediate film layer; 5 represents an electrochemical pump; 51 represents a pump body; 52 represents an electrolyte solution; 53 represents an electrode layer; 54 represents an expanded film; A represents an accommodation zone; B represents an injection channel.

### DETAILED DESCRIPTION OF THE EMBODIMENTS

The following will provide a clear and complete description of the technical solution in embodiments of the present disclosure, in conjunction with the accompanying drawings. Obviously, the described embodiments are only a part of the embodiments of the present disclosure, not all of them. Based on the embodiments in the present disclosure, all other embodiments obtained by ordinary technicians in the art without creative labor shall fall within the scope of the present disclosure.

An object of the present disclosure is to provide a diabetes sensor, a method for manufacturing the diabetes sensor and a closed-loop control system, to solve the problems existing in the prior art, and to improve the comfort level when using microneedles and further reduce the risk of infection.

In order to make the above object, features, and advantages of the present disclosure more apparent and understandable, the following will provide further detailed descriptions of the present disclosure in conjunction with the accompanying drawings and specific embodiments.

### Technical Solution 1: A Diabetes Sensor and Manufacturing Method Thereof

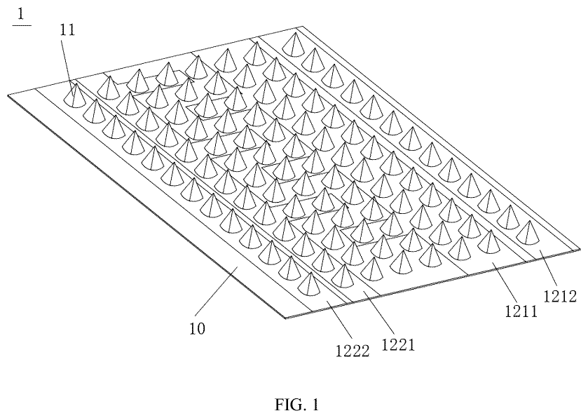

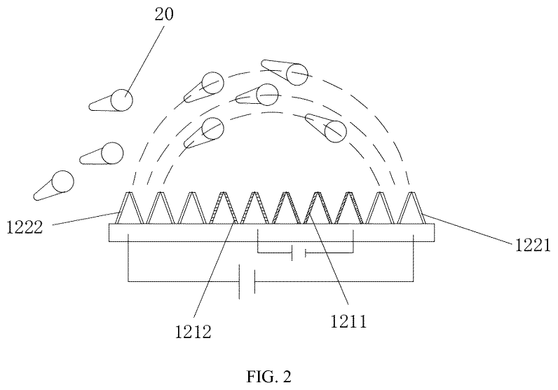

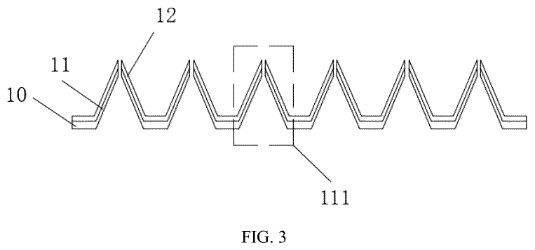

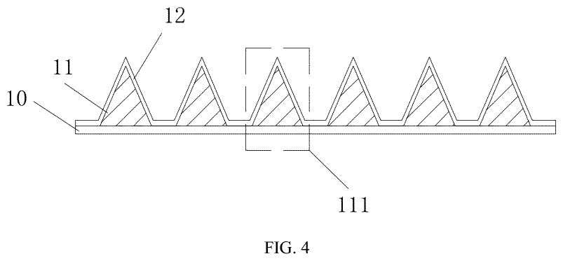

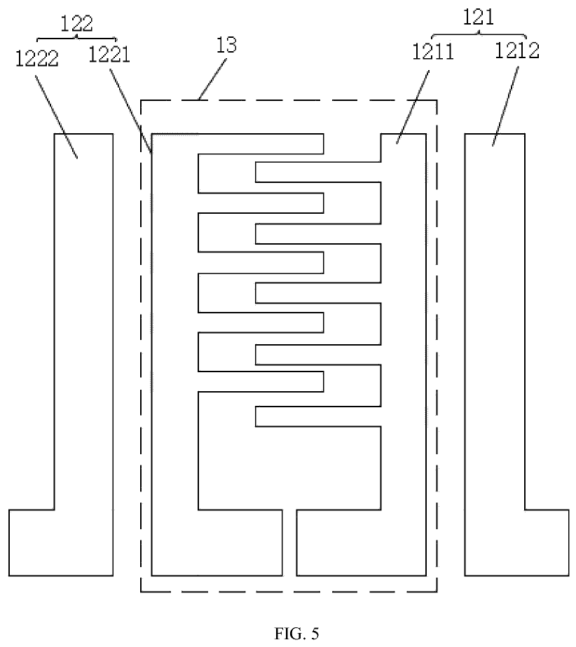

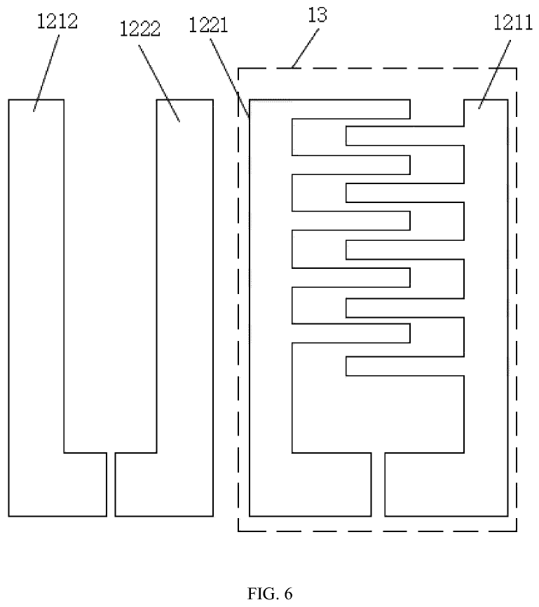

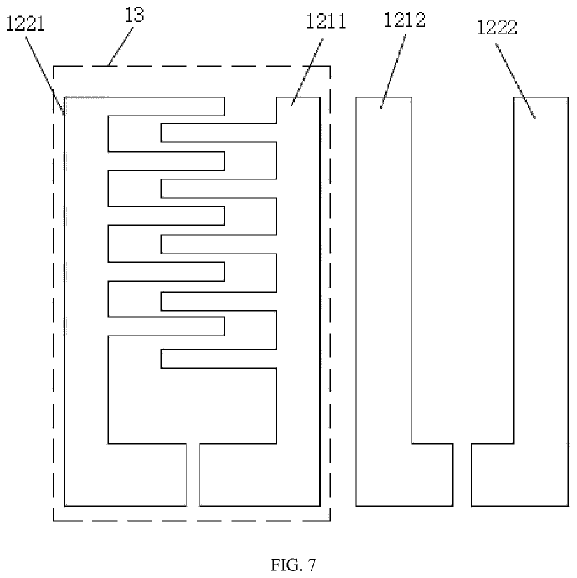

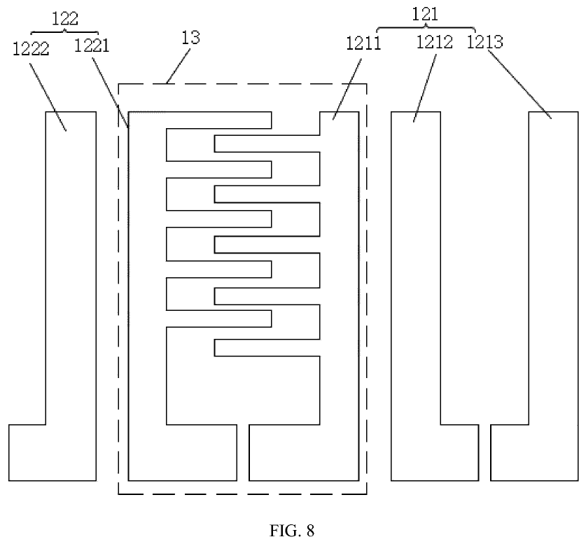

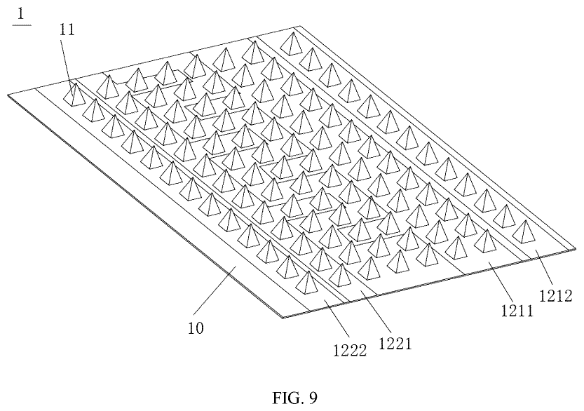

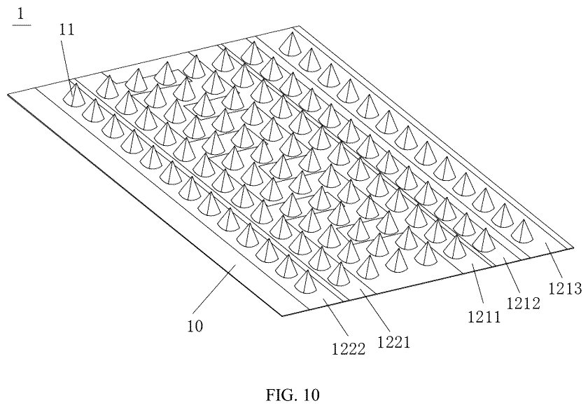

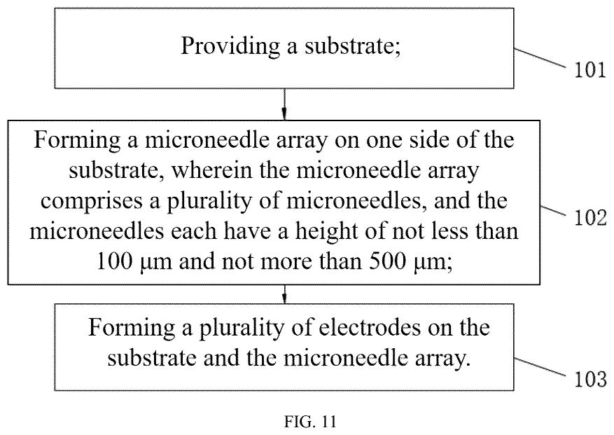

Referring to FIG. 1, FIG. 2, FIG. 3, FIG. 4, FIG. 5, FIG. 6, FIG. 7, FIG. 8, FIG. 9, FIG. 10 and FIG. 11, a diabetes sensor is provided in this technical solution of the disclosure. The diabetes sensor includes a substrate 10, a microneedle array 11 arranged on one side of the substrate 10, and a plurality of electrodes 12 covering the substrate 10.

In some embodiments, the substrate 10 and the microneedle array 11 could be integrally formed, that is to say, the substrate 10 and the microneedle array 11 could be manufactured by the same method or the same step. The microneedle array 11 includes a plurality of microneedles 111, which are cones or pyramids with a certain height (see FIGS. 1 and 8). It should be noted that the height refers to the size of microneedles 111 in the direction perpendicular to the substrate 10.

In some embodiments of the disclosure, the microneedles 111 each have a height of not less than 100 μm, and not more than 1000 μm, for example, a height of 100 μm, 200 μm, 300 μm, 400 μm, 500 μm, 600 μm, 700 μm, 800 μm, 900 μm, 1000 μm and so on. When using the diabetes sensor, the microneedle array 11 needs to be inserted into the patient's body, so the microneedles 111 with a shorter height could reduce the pain feeling of the patient.

In some embodiments of the present disclosure, the substrate 10 and the microneedle array 11 are each independently made of a polymeric material, a biodegradable material, or a biocompatible material. For example, when the substrate 10 and microneedle array 11 are made of a polymeric material, the substrate 10 and microneedle array 11 are made of Si; when the substrate 10 and microneedle array 11 are made of a biodegradable material, the substrate 10 and microneedle array 11 are made of chitosan or polylactic acid; when the substrate 10 and microneedle array 11 are made of a biocompatible material, the substrate 10 and microneedle array 11 are made of a thermoplastic polyurethane, etc. Due to the possible rupture of microneedles 111 after the microneedle array 11 is inserted into the patient's body, the use of the above materials could avoid damage to the human body caused by the ruptured microneedles 111 remaining in the patient's body, while also reduce the risk of infection.

As shown in FIGS. 3 and 4, in some embodiments of the present disclosure, the microneedle array 11 includes a solid microneedle array or a hollow microneedle array.

In some embodiments, as shown in FIG. 3, the microneedles 111 of the hollow microneedle array are hollow cones or pyramids with internal channels through which interstitial fluid from the patient could enter the microneedles 111. Meanwhile, insulin injection could be achieved by utilizing a hollow microneedle array. In some embodiments, insulin is stored in an injection pump beforehand and injected into the patient's body through the hollow microneedles 111 using the injection pump.

As shown in FIG. 4, in some embodiments, the microneedles 111 of the solid microneedle array are solid cones or pyramids, and the interstitial fluid from the patient's body could not enter the microneedles.

In some embodiments, as shown in FIGS. 5 and 8, the plurality of electrodes 12 includes an electrochemical sensor 121 and a reverse iontophoresis device 122, wherein the electrochemical sensor 121 includes a working electrode 1211 and a counter electrode 1212; or, the electrochemical sensor 121 includes working electrode 1211, reference electrode 1213, and counter electrode 1212; and the reverse iontophoresis device 122 includes a positive electrode 1222 and a negative electrode 1221.

In the disclosure, the electrochemical sensor could be used to detect glucose molecules in human interstitial fluid and generate electrical signals, and the reverse iontophoresis device 122 could generate reverse iontophoresis effect to attract the glucose molecules from the deep skin layer to an upper part of dermis where the needle tips of the microneedles 111 are located.

In some embodiments, glucose oxidase is immobilized on working electrode 1211. When working electrode 1211 comes into contact with the interstitial fluid in the patient's body, glucose oxidase reacts with the glucose contained in the patient's interstitial fluid and generates products through a glucose oxidase reaction. The products further undergoes oxidation or reduction reaction(s) on the working electrode 1211, resulting in changes in electrical signals. The working electrode 1211 could be made of carbon, gold, platinum, a carbon composite material, a gold composite material, a platinum composite material, or silver/silver chloride.

In some embodiments, a liquid biocompatible polymer covers the working electrode 1211, and the liquid biocompatible polymer is heated and dried to form a biocompatible polymer layer. In some embodiments, perfluorosulfonic acid is selected as a material for forming the biocompatible polymer layer. The biocompatible polymer layer could avoid damage to the human body caused by the prussian blue layer included in the working electrode.

In some embodiments, referring to FIG. 5, FIG. 8 and FIG. 10, when the electrochemical sensor only includes a working electrode 1211 and a counter electrode 1212, the counter electrode 1212 plays the role of closing the circuit and while stabilizing the voltage in the electrochemical sensor 121, and silver/silver chloride is selected as a material for the counter electrode 1212. As shown in FIG. 5, in some embodiments, the electrochemical sensor 121 includes a working electrode 1211, a reference electrode 1213 and a counter electrode 1212, and the reference electrode 1213 plays a role of stabilizing the voltage in the electrochemical sensor 121, and the counter electrode 1212 plays a role of closing the circuit in the electrochemical sensor 121. In some embodiments, the electrode 1212 is made of a material selected from the group consisting of gold, platinum, carbon, a gold composite material, a platinum composite material, and a carbon composite material. In some embodiments, silver/silver chloride is selected as a material for reference electrode 1213.

As shown in FIG. 5, in some embodiments, the reverse iontophoresis device 122 is arranged on one side of the working electrode 1211. The reverse iontophoresis device 122 includes a negative electrode 1221 and a positive electrode 1222, and the negative electrode 1221 and the working electrode 1211 form the interdigital electrodes 13. In some embodiments, the counter electrode 1212 of the electrochemical sensor 121 and the positive electrode 1222 of the reverse iontophoresis device 122 are located on one or both sides of the interdigital electrodes.

In some embodiments, the reverse iontophoresis device 122 is made of a material including silver/silver chloride, silicone material, a conductive polymer, graphene, and gold. In some embodiments, the negative electrode 1221 and positive electrode 1222 of the reverse iontophoresis device 122 are made of the same material or different materials. For example, both of the negative electrode 1221 and positive electrode 1222 of the reverse iontophoresis device 122 are made of silver/silver chloride.

In some embodiments, as shown in FIG. 6, the counter electrode 1212 and the positive electrode 1222 are simultaneously located on the left side of the interdigital electrodes 13. Alternatively, as shown in FIG. 7, in some embodiments, the counter electrode 1212 and the positive electrode 1222 are simultaneously located on the right side of the interdigital electrodes 13. Alternatively, in some embodiments, the positive electrode 1222 is located on the left side of the interdigital electrodes 13, and the counter electrode 1212 is located on the right side of the interdigital electrodes 13.

As shown in FIG. 2, when using the above diabetes sensor, the reverse iontophoresis device is configured to attract glucose molecules 20 from the deep subcutaneous area to the shallow painless epidermal area, which promotes the diffusion and transmission of glucose molecules 20, which effectively increases the movement speed and aggregation degree of glucose molecules 20 in the skin, thus improving the sensitivity and accuracy of glucose detection.

In some embodiments, the reverse iontophoresis device 122 could generate reverse iontophoresis effect on the interstitial fluid in the patient's body, thereby attracting glucose to move to the position of the reverse iontophoresis device 122. Due to the fact that the negative electrode 1221 of the reverse iontophoresis device 122 and the working electrode 1211 form the interdigital electrodes 13, glucose thus could move to the position of the working electrode 1211, such that the working electrode 1211 could more efficiently detect glucose in the patient's body, thereby improving detection efficiency of the diabetes sensor.

In the diabetes sensor of the disclosure, in the first aspect, in some embodiments, the microneedle array 11 contains microneedles 111 each having a height of 100 μm to 1000 μm, which could alleviate the pain caused to patients. In the second aspect, interdigital electrodes are formed between the working electrode 1211 and the negative electrode 1221 of the reverse iontophoresis device 122, and the reverse iontophoresis device 122 is configured to attract glucose in the patient's interstitial fluid to the position of the interdigital electrodes 13, thereby improving the efficiency of the electrochemical sensor 121 in detecting glucose, and further making the use of the diabetes sensor more convenient.

Meanwhile, compared with the traditional reverse iontophoresis on the skin surface, the efficiency of reverse iontophoresis is further enhanced as the microneedles penetrate the high-resistance epidermal layer, which attracts more glucose molecules to the vicinity of the electrode, thereby improving the accuracy and reliability of the diabetes sensor. In some embodiments, the height of the microneedles included in the microneedle array could be reduced to 100-1000 μm, such that the microneedles having such height do not reach the nerve layer, thereby reducing pain and infection risk. In addition, compared with the traditional microneedle methods, the diabetes sensor of the disclosure reduces the depth to which the microneedles are inserted into the skin with proviso of meeting the accuracy, precision, and requirements, thereby further reducing the pain feeling.

Based on the same inventive concept, the disclosure also provides an insulin injection system, including any kind of a diabetes sensor as described previously in embodiments of the disclosure, a control module, and an injection pump.

In some embodiments, an input end of the control module is connected with an output end of the diabetes sensor, and an output end of the control module is connected with an input end of the syringe pump. Therefore, the control module could detect and receive the electrical signal output by the diabetes sensor. Since the microneedles 111 enter the patient's body and contact the patient's subcutaneous interstitial fluid, the glucose concentration of the patient's subcutaneous interstitial fluid therefore could be measured. Further, the glucose concentration of the interstitial fluid has a strong correlation with the blood glucose concentration, so the electrical signal output by the diabetes sensor could reflect the blood glucose concentration. For example, the diabetes sensor could detect a current at a constant voltage, and the intensity of the current signal is directly proportional to the level of the glucose concentration.

The control module could control opening or closing of the injection pump according to the electrical signal, that is to say, to turn on or off the injection pump. For example, a preset value could be set in the control module. If the value of the electrical signal is not less than the preset value, the injection pump would be powered on. If the value of the electrical signal is less than the preset value, the injection pump would not be powered on.

In this way, the injection pump could be controlled according to the patient's real-time blood glucose concentration, and an insulin solution could be injected into the patient's body through the microneedle array 11.

In specific embodiments, the control module could be a controller or a control chip.

FIG. 11 shows a flowchart of a method for manufacturing the diabetes sensor. Referring to FIG. 10, the disclosure also discloses a method for manufacturing the diabetes sensor, which includes step 101, providing a substrate 10; step 102, forming a microneedle array 11 on one side of the substrate 10; and step 103, forming a plurality of electrodes 12 on the substrate 10 and the microneedle array 11.

### Regarding Step 101:

In some embodiments, the substrate 10 is integrally formed with the microneedle array 11, or the substrate 10 is formed first and the microneedle array 11 is then formed. In some embodiments, the substrate 10 and the microneedle array 11 are made of the same material, such that the substrate 10 and the microneedle array 11 are better fused.

### Regarding Step 102:

In some embodiments, the microneedle array 11 includes a plurality of microneedles 111, and the microneedles 111 each have a height of not less than 100 μm and not more than 1000 μm.

In some embodiments, the step of forming the microneedle array 11 on one side of the substrate 10 includes step 1021, providing a mold with a microneedle sequence that matches the microneedle array 11; and step 1022, pouring a polymeric material into the mold, solidifying the polymeric material, and then peeling a resulting solidified polymeric material off the mold, to obtain the microneedle array 11.

### Regarding Step 1021:

In some embodiments, the mold is fabricated by soft lithography. In some embodiments, the mold is made of polydimethylsiloxane (PDMS). The pattern of the microneedle sequence in the mold matches the pattern of the microneedle array 11. It can be understood that the microneedle array 11 could be formed by using the microneedle sequence. In some embodiments, the microneedles 111 in the microneedle array each have a height of not less than 100 μm and not more than 1000 μm.

### Regarding Step 1022:

In some embodiments, the polymeric material includes chitosan or polylactic acid. Those skilled in the art could choose according to their needs. After being poured into the mold, the polymeric material is solidified, and dried. In some embodiments, the drying is performed at 60° C. In some embodiments, the drying is performed for 30 min. Then after the polymeric material is solidified, the solidified polymeric material is peeled off the mold, to obtain the microneedle array 11.

It should be noted that in this method, the substrate 10 and the microneedle array 11 are integrally formed, such that the substrate 10 is fabricated while obtaining the microneedle array 11.

In some embodiments, the microneedle array 11 is fabricated on the substrate by a 3D printing process or micro/nanofabrication process. In some embodiments, the substrate 10 is fabricated in advance.

In some embodiments, 3D printing is usually realized by using digital printers. First, the 3D model is constructed by a computer modeling software, and then the built 3D model is “partitioned” into layer-by-layer sections, i.e., slices, so as to guide the printer to print layer by layer according to slices.

Specific steps of the micro/nanofabrication technology include template processing, pattern transfer and substrate processing. Among them, template processing generally uses reactive ion etching and other means to process the required microneedle structure on silicon or other substrates as a template. Pattern transfer refers to applying a soft material onto the surface of the template, and a template is then pressed on the surface to transfer the pattern to the soft material mold. Substrate processing refers to applying a desired material onto the soft mold and solidifying the material. After removing the template, a high-precision processed material is finally obtained.

### Regarding Step 103:

In some embodiments, the plurality of electrodes 12 includes an electrochemical sensor 121 and a reverse iontophoresis device 122, wherein the electrochemical sensor 121 includes a working electrode 1211 and a counter electrode 1212, or includes a working electrode 1211, a reference electrode 1213, and a counter electrode 1212; and the reverse iontophoresis device 122 includes a positive electrode 1222 and a negative electrode 1221.

The working electrode 1211 of the electrochemical sensor 121 and the negative electrode 1221 of the reverse iontophoresis device 122 form the interdigital electrodes 13; and the counter electrode 1212 of the electrochemical sensor 121 and the positive electrode 1222 of the reverse iontophoresis device 122 are located on one or two sides of the interdigital electrodes 13.

In some embodiments, glucose oxidase is immobilized on the working electrode 1211 of the electrochemical sensor 121. The electrochemical sensor 121 is configured to detect glucose in interstitial fluid and generate an electrical signal, and the reverse iontophoresis device 122 is configured to generate a reverse iontophoresis effect to attract glucose molecules from a deep skin layer to an upper part of the dermis where the needle tips of the microneedles 111 are located.

In specific embodiments, the plurality of electrodes 12 is formed by a micro/nanofabrication process, a screen printing process, or an aerosol jet printing process, which could be selected by those skilled in the art according to actual needs.

In the diabetes sensor manufactured by the above method, in a first aspect, the microneedle array 11 includes microneedles 111 each having a height of 100 μm to 1000 μm, which could reduce the pain caused to patients; in a second aspect, the working electrode 1211 and the negative electrode 1221 of the reverse iontophoresis device 122 form the interdigital electrodes. The reverse iontophoresis device 122 is configured to attract glucose in the patient's interstitial fluid to the location of the interdigital electrodes, such that the efficiency of the electrochemical sensor 121 in detecting glucose is improved, and the use of the diabetes sensor is thereby more convenient.

Meanwhile, compared with the traditional reverse iontophoresis on the skin surface, the strength of reverse iontophoresis is further enhanced as the microneedles penetrate the high-resistance epidermal layer, which attracts more glucose molecules to reach the vicinity of the electrode, thereby improving the accuracy and reliability of the diabetes sensor. The microneedles having such height do not reach the nerve layer, thereby reducing pain and infection risk. Moreover, compared with the traditional microneedle methods, the diabetes sensor according to the disclosure reduces the depth to which the microneedles are inserted into the skin with proviso of meeting the accuracy, precision, and requirements, thereby further reducing the pain feeling.

In the present disclosure, the process for manufacturing the microneedle array and the process for manufacturing the electrode could be randomly combined.

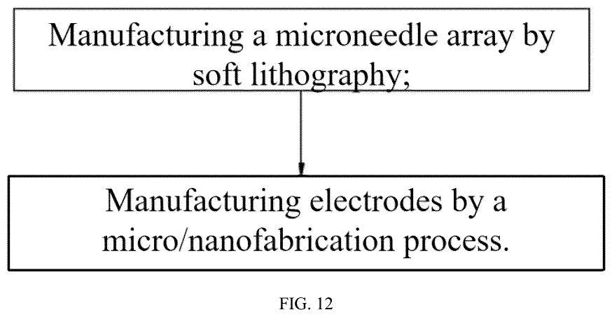

For example, as shown in FIG. 12, the microneedle array 11 is fabricated by soft lithography (i.e., the process described in steps 1021 and 1022), and the electrodes 12 are then fabricated by the micro/nanofabrication process.

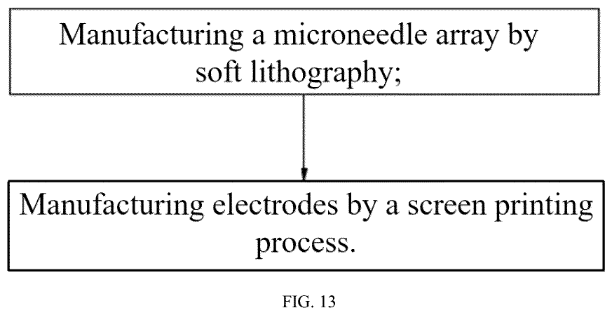

For example, as shown in FIG. 13, the microneedle array 11 is fabricated by soft lithography (i.e., the process described in steps 1021 and 1022), and the electrodes 12 are then fabricated by the screen printing process.

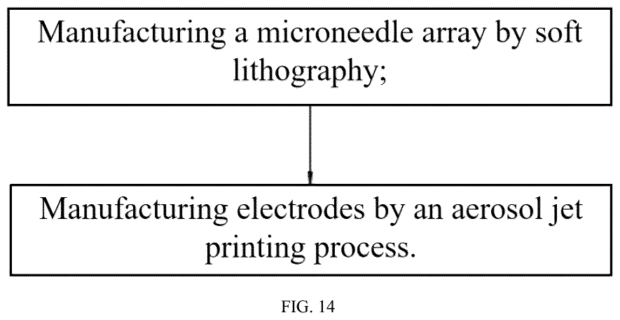

For example, as shown in FIG. 14, the microneedle array 11 is fabricated by soft lithography (i.e., the process described in steps 1021 and 1022), and the electrodes 12 are then fabricated by the aerosol jet printing process.

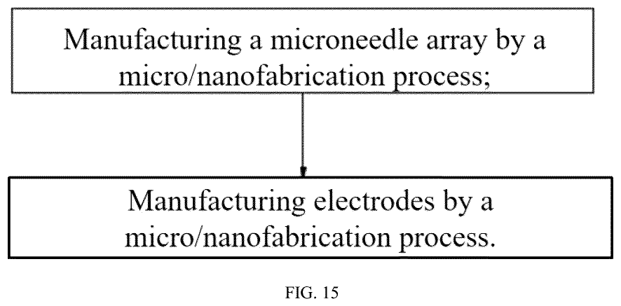

For example, as shown in FIG. 15, the microneedle array 11 is fabricated by the 3D printing process, and the electrodes 12 are then fabricated by the micro/nanofabrication process.

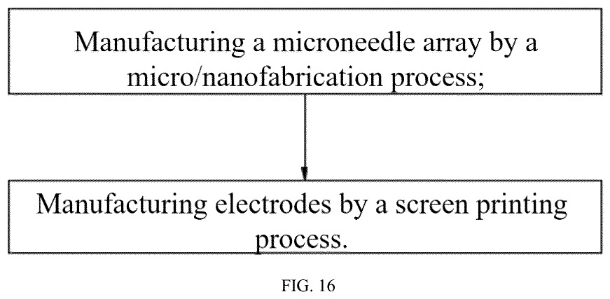

For example, as shown in FIG. 16, the microneedle array 11 is fabricated by the 3D printing process, and the electrodes 12 are then fabricated by the screen printing process.

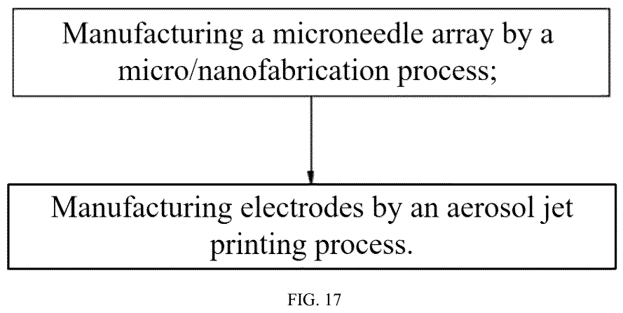

For example, as shown in FIG. 17, the microneedle array 11 is fabricated by the 3D printing process, and the electrodes 12 are then fabricated by the aerosol jet printing process.

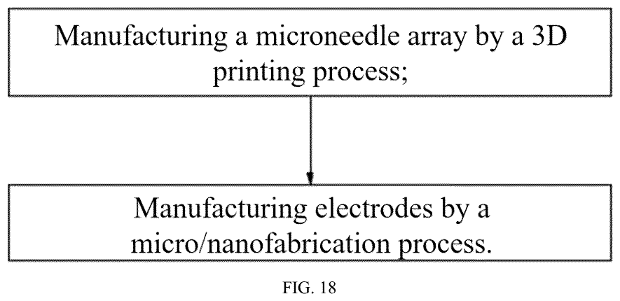

For example, as shown in FIG. 18, the microneedle array 11 is fabricated by the micro/nanofabrication process, and the electrodes 12 are then fabricated by the micro/nanofabrication process.

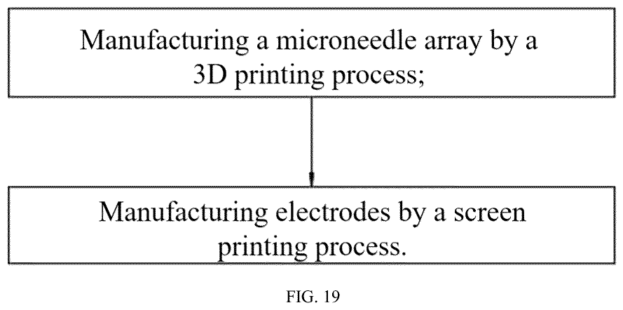

For example, as shown in FIG. 19, the microneedle array 11 is fabricated by the micro/nanofabrication process, and the electrodes 12 are then fabricated by the screen printing technology.

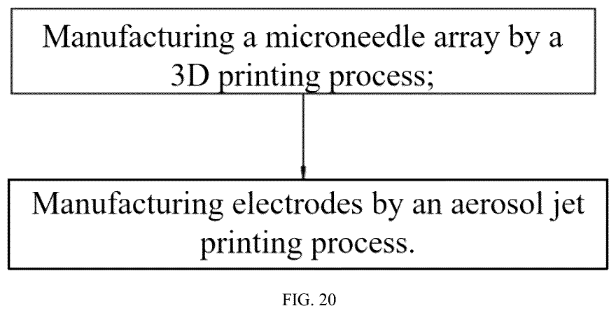

For example, as shown in FIG. 20, the microneedle array 11 is fabricated by the micro/nanofabrication process, and the electrodes 12 are then fabricated by the aerosol jet printing process.

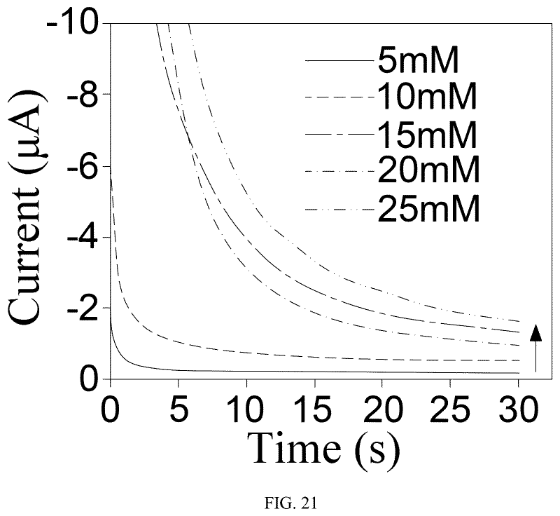

In some embodiments of the disclosure, experiments were conducted on the diabetic sensors with applying reverse iontophoresis current (by using the reverse iontophoresis device) and without applying reverse iontophoresis current. Result data obtained is shown in FIGS. 21 to 36.

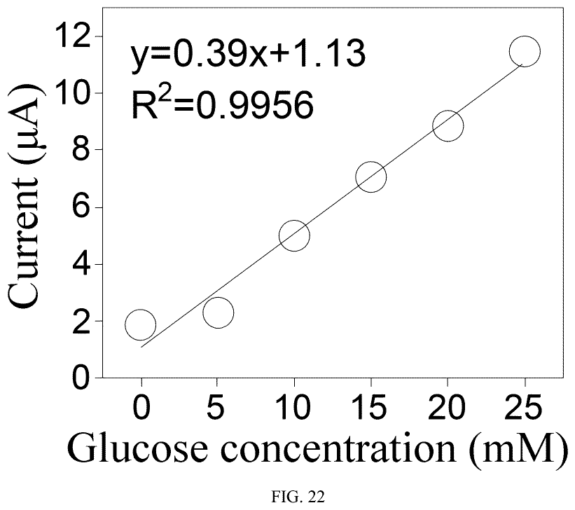

FIG. 21 is a curve chart showing current response of the diabetes sensor including microneedles of 400 μm in height to different glucose concentrations, after applying a reverse iontophoresis current of 0.3 mA for 200 s. According to the data obtained in FIG. 21, the sensitivity of the diabetes sensor is calculated to be 0.39 μA/mM (as shown in FIG. 22).

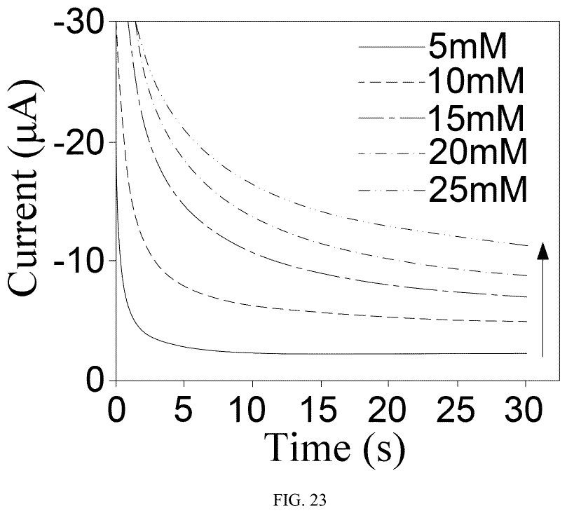

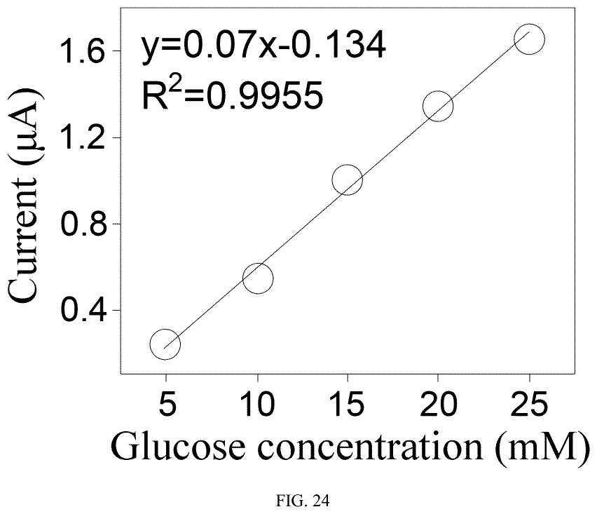

FIG. 23 is a curve chart showing current response of the diabetes sensor to different glucose concentrations, without applying reverse iontophoretic current. According to the data obtained in FIG. 23, it can be calculated that the sensitivity of the diabetes sensor is 0.07 μA/mM (as shown in FIG. 24). Therefore, it can be seen that the sensitivity of the diabetes sensor could be improved at least five times by applying a reverse iontophoresis current (i.e., adding the reverse iontophoresis device).

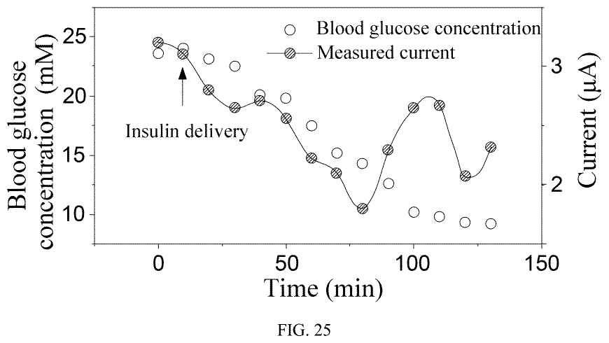

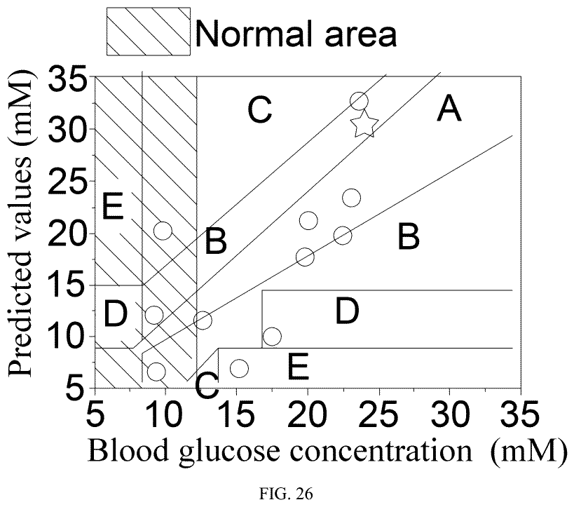

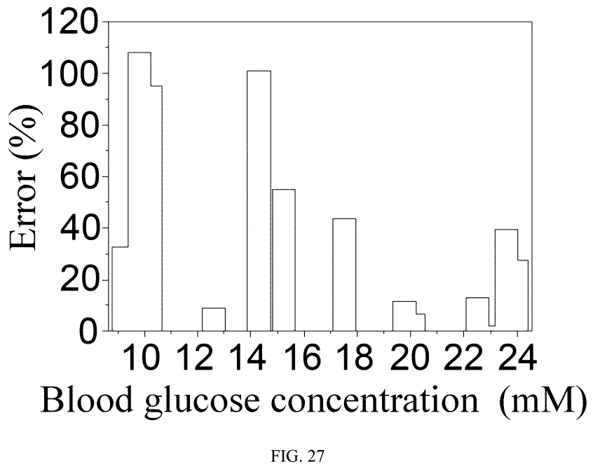

FIG. 25 shows the trends of the current variations of the diabetes sensor without reverse iontophoresis and blood glucose variations of rat after subcutaneous injection of insulin into diabetic rats. The experimental results show that the currents deviate from rat blood glucose, and some of the predicted values are distributed in areas D and E (as shown in FIG. 26). Such error would lead to excessive underestimation or overestimation of blood glucose concentration, thus causing a risk of serious hyperglycemia or hypoglycemia. Also, the average error when not applying reverse iontophoresis is 40%±36%, indicating that the diabetes sensor has low accuracy (as shown in FIG. 27).

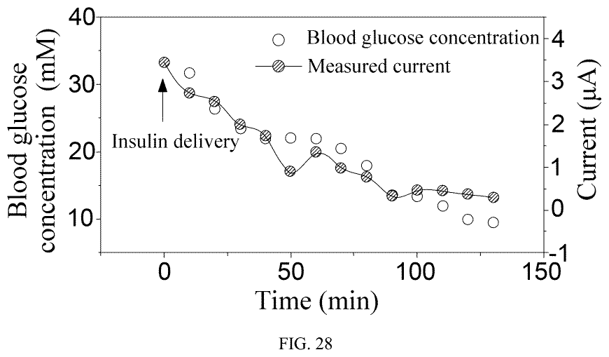

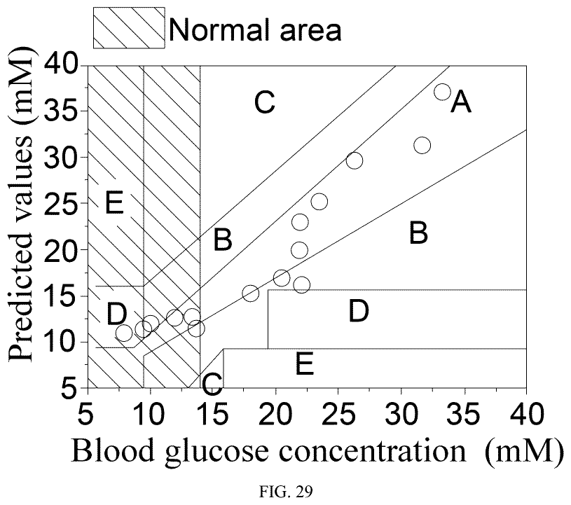

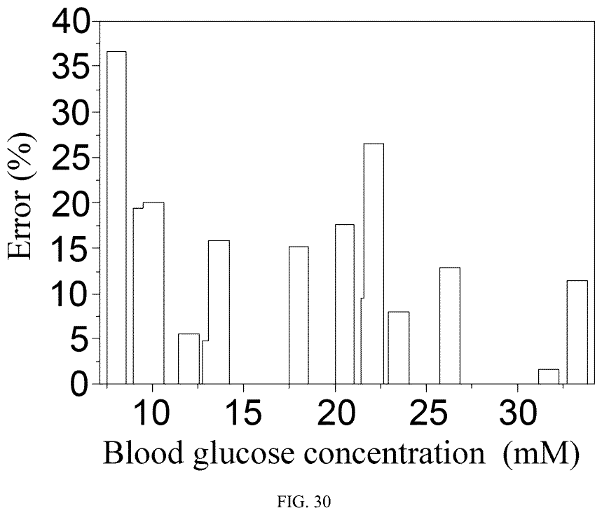

FIG. 28 shows the trends of the current variations of the diabetic sensor with reverse iontophoresis after insulin injection into diabetic rats. As can be seen, the trends of current changes and the blood glucose levels are substantially consistent, 93% of the predicted points are distributed in area A and area B, which substantially would not cause false medical diagnosis (as shown in FIG. 29), the detection error being 18.1%±11.3% (as shown in FIG. 30).

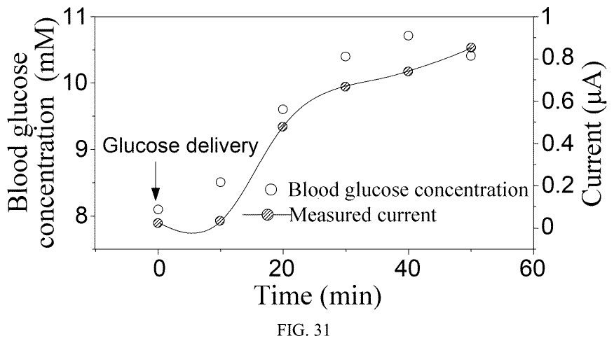

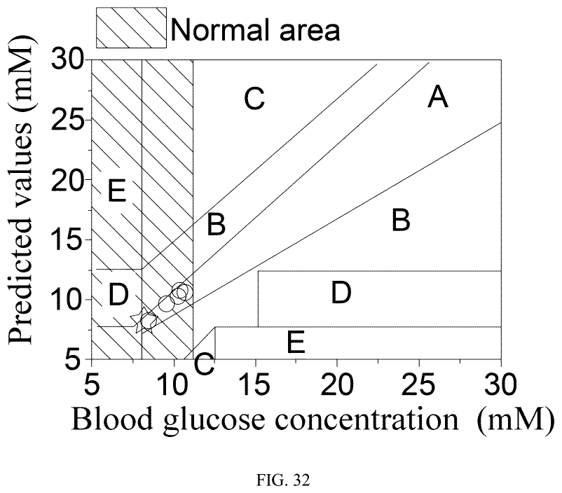

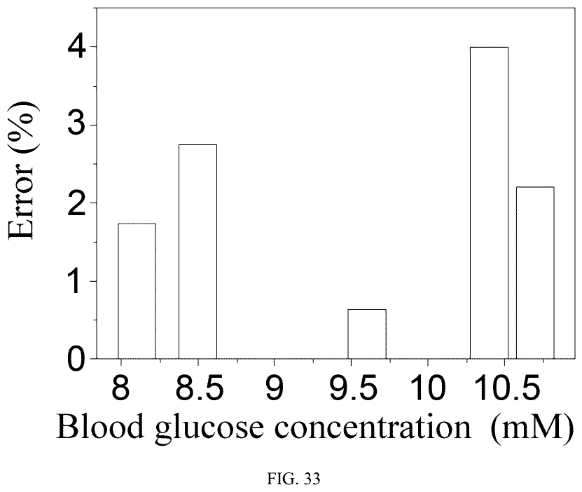

FIG. 31 shows the trends of the current variations of the diabetic sensor with reverse iontophoresis after injecting glucose into healthy rats. The trend of blood glucose variations could substantially be judged according to the current variation. All predicted points are distributed in area A of the Clarke error grid (as shown in FIG. 32), with an average error percentage of 2% #1% (as shown in FIG. 33). It shows that the diabetes sensor with reverse iontophoresis exhibits a high accuracy in predicting a rising current. On the other hand, due to blood glucose variations within a small range (i.e., 8-11 mM) and lower concentration, it results in more accurate measurement compared with a falling current.

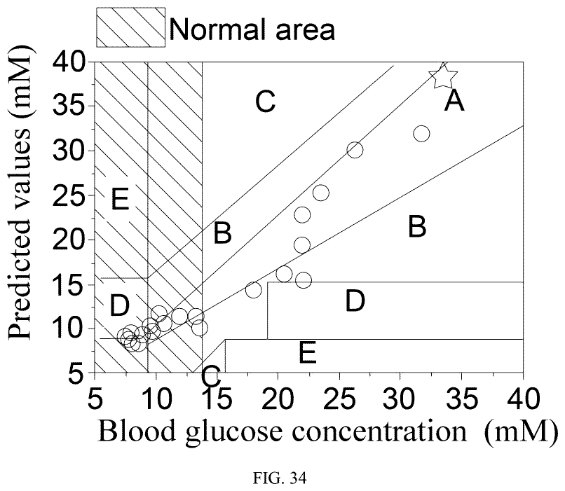

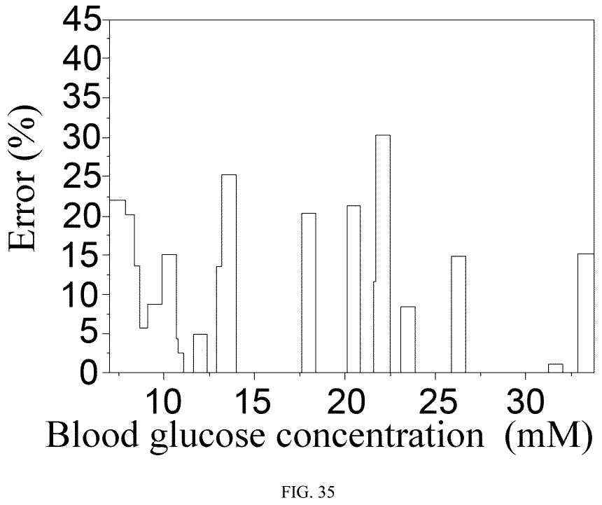

FIG. 34 and FIG. 35 show the comparison between the accurate values and the values measured by the diabetes sensor with reverse iontophoresis (summarized from FIG. 28 and FIG. 33) and the error percentages (10.98%±8.15%), respectively. FIG. 35 shows the comparison of blood glucose prediction error percentages before and after using reverse iontophoresis. It is found that after the enrichment of glucose molecules by electroosmosis, the error percentage of the diabetes sensor is reduced by 3 times compared with those without reverse iontophoresis, which indicates the improvement of the accuracy for the short microneedle subcutaneous sensing by reverse iontophoresis.

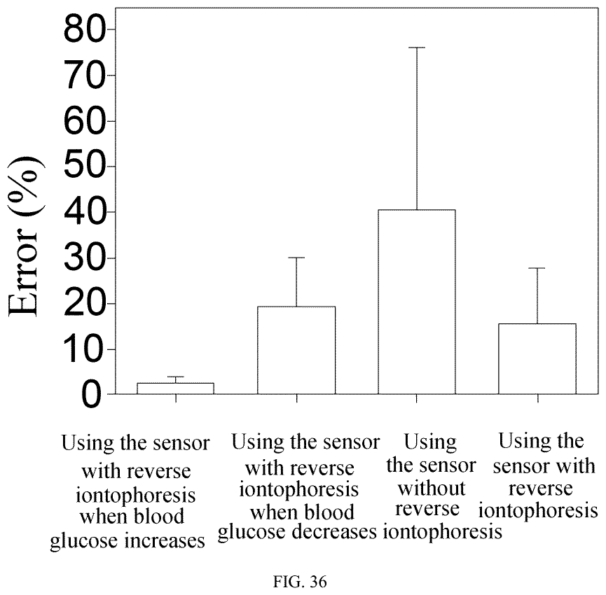

FIG. 36 shows the comparison of blood glucose prediction error percentages before and after the use of reverse iontophoresis. It is found that after the enrichment of glucose molecules by reverse iontophoresis, the error percentage of the diabetes sensor is reduced by 3 times compared with those without reverse iontophoresis, which indicates the improvement of the accuracy for short microneedles subcutaneous sensing by reverse iontophoresis.

Technical Solution 2: A Closed-Loop Control System with an Ultrasonic Pump

Among related technologies, ultrasonic pump is a new device for insulin injection. When using ultrasonic pump as an insulin pump for quantitative injection of insulin for patients, it is necessary to control the switch of the ultrasonic pump according to the blood glucose concentration in patients. However, at present, there is no relative mature solution for the control of the ultrasonic pump.

In view of this, this technical solution of the present disclosure provides a closed-loop control system, in which an ultrasonic pump, a diabetes sensor, and a control module are configured, wherein a liquid outlet of the ultrasonic pump is connected with the substrate of the diabetes sensor; an input end of the control module is connected with an output end of the diabetes sensor; and an output end of the control module is connected with an input end of the ultrasonic pump. The glucose concentration in subcutaneous interstitial fluid of patients could be measured using the diabetes sensor. Because the glucose concentration in interstitial fluid is greatly correlated with the blood glucose concentration, the signal output by the diabetes sensor could reflect the blood glucose concentration. Also, the diabetes sensor could output signals to the control module, and the control module then could control the opening or closing of the ultrasonic pump according to the electrical signal output by the diabetes sensor, such that the ultrasonic pump could inject insulin according to the blood glucose concentration of the patient in real time.

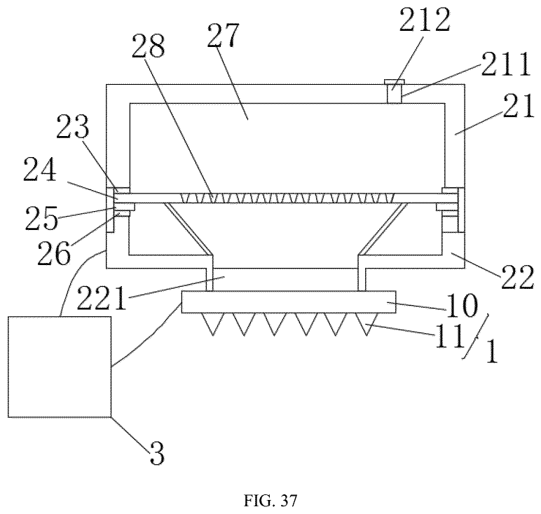

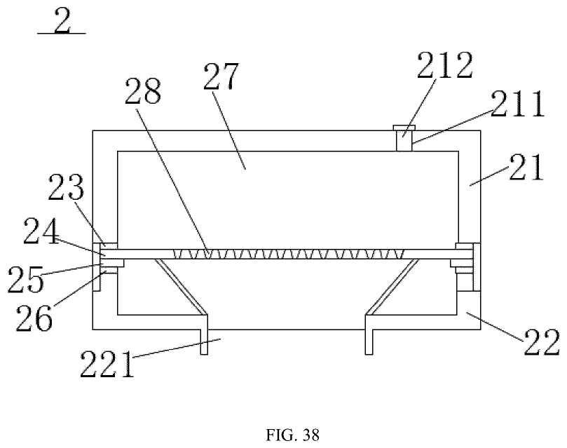

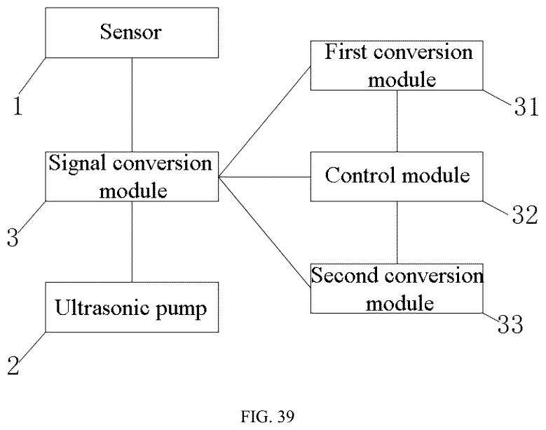

FIG. 37, FIG. 38 and FIG. 39 show a closed-loop control system according to embodiments of the disclosure. The closed-loop control system includes an ultrasonic pump 2, a diabetes sensor 1, and a signal conversion module 3.

In some embodiments, as shown in FIG. 38, the ultrasonic pump 2 includes an upper casing 21 and a lower casing 22, as well as a first sealing ring 23, a thin film 24, a piezoelectric ring 25, and a second sealing ring 26 arranged from up to bottom between the upper casing 21 and the lower casing 22.

In some embodiments, the thin film 24 is made of a metal, a rubber or an elastomer material, specifically being one selected from stainless steel, gold, copper, zinc, platinum, silver, tungsten, aluminum, an aluminum alloy, natural rubber, isoprene rubber, polybutadiene rubber, styrene butadiene rubber, nitrile rubber, chloroprene rubber, butyl rubber, halogenated butyl rubber, ethylene propylene rubber, ethylene propylene diene rubber, epichlorohydrin rubber, polyacrylate rubber, silicone rubber, fluorosilicone rubber, fluororubber, chlorosulfonated polyethylene, hydrogenated nitrile rubber, thermoplastic polyolefin elastomer, thermoplastic styrene elastomer, polyurethane thermoplastic elastomer, polyester thermoplastic elastomer, polyamide thermoplastic elastomer, halogen containing thermoplastic elastomer, ionic thermoplastic elastomer, ethylene copolymer thermoplastic elastomer, 1,2-poly-butadiene thermoplastic elastomer, trans polyisoprene thermoplastic elastomer, melt processible thermoplastic elastomer, thermoplastic vulcanizate, and polydimethylsiloxane. In some embodiments, the thin film 24 is made of a hard material or flexible material. In this solution, the flexible material is preferred, such that the thin film 24 has certain flexibility and can be stretched and bent well.

As shown in FIG. 38, in some embodiments, a plurality of conical holes 28 are distributed on the thin film 24. The large-diameter end of the conical holes 28 are each located on the side of the upper casing 21, and the small-diameter end thereof is located on the side of the lower casing 22. In specific embodiments, the conical holes 28 at the large-diameter end each have a diameter larger than the molecular diameter of insulin, and the conical holes 28 at the small-diameter end each have a diameter smaller than the molecular diameter of insulin, such that insulin could enter the conical holes 28 but could not flow out automatically. In some embodiments, the conical holes 28 are formed by laser etching or ion selective etching, which are convenient to form, and have high hole-diameter accuracy, and smooth inner surface to facilitate the flow of insulin in the holes.

As shown in FIG. 38, the space between the thin film 24 and the upper casing 21 constitutes the drug storage chamber 27. The drug storage chamber 27 is configured to store the drug to be injected. In some embodiments, the drug stored in the drug storage chamber 27 is an insulin injection solution having a concentration of 1-1000 U/ml, which is used to supplement insulin injection for patients developing diabetes.

As shown in FIG. 38, a piezoelectric ring 25 is arranged on the side of the thin film 24 facing away from the drug storage chamber 27. In some embodiments, the piezoelectric ring 25 is made of piezoelectric materials such as a piezoelectric crystal, a piezoelectric ceramic, and a piezoelectric polymer. In some embodiments, the edges on sides opposite to each other of the thin film 24 and the piezoelectric ring 25 are respectively connected to an external alternating-current power supply through conductive wire(s). In some embodiments, the external alternating-current power supply has a voltage of 10-100 V. Alternatively, in some embodiments, they are respectively connected to a direct current power supply having a voltage of 1-10 V, which is converted to 10-100 V alternating current through a circuit.

When the piezoelectric ring 25 is connected to an alternating current, it undergoes radial vibration, thus driving the thin film 24 to vibrate. The thin film 24 repeatedly extends and bends during vibration, such that the conical holes 28 stretch, and the hole diameters thereof change. That is to say, the large-diameter end and small-diameter end of the conical holes 28 change repeatedly, the diameter at the large-diameter end becomes smaller, and the diameter at the small-diameter end becomes larger, so as to extrude the drug stored in the drug storage chamber 27 downward from the conical holes 28, and finally make the drug flow out of the liquid outlet 221, thereby achieving the effect of vibration drug delivery. In addition, when the piezoelectric ring 25 is connected to the alternating current, it undergoes radial vibration and meanwhile ultrasound is generated, which could promote the human body to absorb insulin.

As shown in FIG. 38, in some embodiments, a first sealing ring 23 is arranged between the thin film 24 and the upper casing 21, and a second sealing ring 26 is arranged between the piezoelectric ring 25 and the lower casing 22. In some embodiments, both the first seal ring 23 and the second seal ring 26 are O-rings, and the first seal ring 23 and the second seal ring 26 are made of a material of rubber, silicone, etc. By using sealing rings, the connection between the parts is tighter, resulting in high tightness, and thus drug leakage would not occur.

As shown in FIG. 38, in some embodiments, the upper casing 21 is also provided with a liquid inlet 211, the liquid inlet 211 is equipped with a rubber plug 212. Plugging the liquid inlet 211 with the rubber plug 212 could prevent impurities from entering the insulin storage chamber to contaminate insulin. After the insulin in the insulin storage chamber is used up, the rubber plug 212 could be pulled out, and new insulin is injected into the insulin storage chamber through the liquid inlet 211, so as to realize continuous use for many times.

As shown in FIG. 37, in some embodiments, the substrate 10 is connected with the liquid outlet 221, and the tip end of the microneedle array 11 faces a side away from the liquid outlet 221.

Specifically, when using the closed-loop control system, the insulin solution is located in the drug storage chamber 27. After powering on the piezoelectric ring 25 of the ultrasonic pump 2, the piezoelectric ring 25 undergoes radial vibration, and drives the thin film 24 to vibrate. The thin film 24 repeatedly extends and bends during vibration, making the conical holes 28 stretch and the hole diameters thereof change. That is to say, the diameters at the large-diameter end and small-diameter end of the conical holes 28 change repeatedly, the diameters thereof at the large-diameter end become smaller, and the diameters thereof at the small-diameter end become larger, so as to extrude the insulin solution stored in the drug storage chamber 27 downward from the conical holes 28, and finally make the drug flow out of the liquid outlet 221. The insulin solution is then injected into the patient through the hollow microneedles 111 on the substrate 10, so as to achieve insulin injection.

Further, the input end of the control module 32 is connected with the output end of the diabetes sensor 1, and the output end thereof is connected with the input end of the ultrasonic pump 2. Therefore, the control module 32 could receive the electrical signal output by the diabetes sensor 1. Since the hollow microneedles 111 on the diabetes sensor 1 enter the patient's body and contact the patient's subcutaneous interstitial fluid, and therefore the glucose concentration in the patient's subcutaneous interstitial fluid could be measured. Further, the glucose concentration in interstitial fluid is highly correlated with the blood glucose concentration, and thus the electrical signal output by the diabetes sensor 1 could reflect the blood glucose concentration. For example, the diabetes sensor 1 could detect a current at a constant voltage, and the level of the current signal is directly proportional to the level of the glucose concentration.

Further, the control module 32 could control the opening or closing of the ultrasonic pump 2 (i.e., powering on or powering off the ultrasonic pump 2) according to the electrical signal. For example, a preset value could be set in the control module 32; if the value of the electrical signal is not less than the preset value, the ultrasonic pump 2 would be powered on; if the value of the electrical signal is less than the preset value, the ultrasonic pump 2 would not be powered on.

In this way, the electrochemical micropump could be controlled according to the real-time blood glucose concentration of patients.

In some embodiments, the substrate and microneedle array of diabetes sensor 1 are fabricated by using a mold in a shape of a microneedle array. In a specific fabrication, a liquid polymeric material is cast in the mold in a shape of microneedle array and demoulded after drying to form the substrate. Among them, in some embodiments, the liquid polymer is a biodegradable material, such as chitosan, polylactic acid, and silk fibroin; the liquid polymer may also be a biocompatible material, such as thermoplastic polyurethane. When the biodegradable material is used, the microneedle diabetes sensor 1 has degradability and could be decomposed naturally after use. The use of the biocompatible material makes the microneedle diabetes sensor 1 more biocompatible and could avoid damage to the human body during use.

In some embodiments, the substrate and microneedle array of diabetes sensor 1 are fabricated by 3D printing. Specifically, the diabetes sensor 1 may be made of a material such as epoxy resin, a ceramic, a metal, a biocompatible material, and a biodegradable material.

As shown in FIG. 7, in some embodiments, the signal conversion module 3 includes a first conversion module 31, a control module 32, and a second conversion module 33. Specifically, the input end of the first conversion module 31 is connected with the output end of the diabetes sensor 1, the output end of the first conversion module 31 is connected with the input end of the control module 32, the input end of the second conversion module 33 is connected with the output end of the control module 32, and the output end of the second conversion module 33 is connected with the input end of the ultrasonic pump 2.

When using the closed-loop control system with the ultrasonic pump, one end of the diabetes sensor 1 enters the patient's body and contacts the patient's subcutaneous interstitial fluid to measure the glucose concentration in the patient's subcutaneous interstitial fluid. Because the glucose concentration of the interstitial fluid is highly correlated with the blood glucose concentration, the signal output by the diabetes sensor 1 could reflect the blood glucose concentration.

Specifically, the diabetes sensor 1 could detect a current at a constant voltage, and the intensity of current signal is directly proportional to the glucose concentration. In addition to detecting the current signal, the first conversion module 31 also provides a constant voltage for the diabetes sensor 1. In some embodiments, the constant voltage is 0.1 V, −0.1 V, or 0.6 V, or other different voltages.

In some embodiments, the diabetes sensor 1 is located outside the patient's body, and the signal conversion module 3 and the ultrasound pump 2 are sequentially provided at one end of the diabetes sensor 1. The ultrasound pump 2 is provided in close proximity to the skin of the patient to realize the injection of insulin for the patient. In addition, the diabetes sensor 1 with a tubular substrate could penetrate deep into the dermis or fat layer of the patient, which is more effective for injecting insulin into the fat layer.

Specifically, the second conversion module 33 could provide a constant voltage to drive the ultrasonic pump and further control the injection dosage of insulin by controlling the voltage level and duration. In some embodiments, the voltage ranges from 0.1 V to 20 V.

In this way, after measuring the glucose concentration and generating an electrical signal by the diabetes sensor 1, the first conversion module 31 of the signal conversion module 3 receives and converts the electrical signal, and then sends the converted electrical signal to the control module 32. After receiving the electrical signal converted by the first conversion module 31, the control module 32 generates different instruction information according to the different electrical signals. For example, the control module 32 generates an opening instruction or a closing instruction, and sends the generated instruction to the second conversion module 33, which converts the received instruction into the corresponding signal, and controls the opening or closing of the ultrasonic pump 2 according to the signal, so as to realize the control of the ultrasonic pump 2 according to the real-time blood glucose concentration of the patient.

In feasible embodiments, the first conversion module 31 is a first signal converter, the control module 32 is a microcontroller, and the second conversion module 33 is a second signal converter.

Specifically, those skilled in the art could utilize devices in the related technology as the first signal converter and the second signal converter, as long as the opening or closing of the ultrasonic pump 2 could be controlled by the control module 32. Therefore, there is no particular limitation on them, and the specific description of the related technology will not be repeated.

In feasible embodiments, the closed-loop control system further includes a cloud server, wherein the control module 32 is electrically connected with the cloud server; and the cloud server is configured to receive and store information sent by the control module 32, the information including a blood glucose concentration in a patient. In feasible embodiments, the closed-loop control system further includes a display module, which is electrically connected with the control module, and meanwhile electrically connected with the cloud server.

The display module is configured to receive and display the information sent by the control module 32. In specific embodiments, the display module is a computer, a display, a tablet computer, etc.

Technical Solution 3: A Closed-Loop Control System with an Electroosmotic Pump

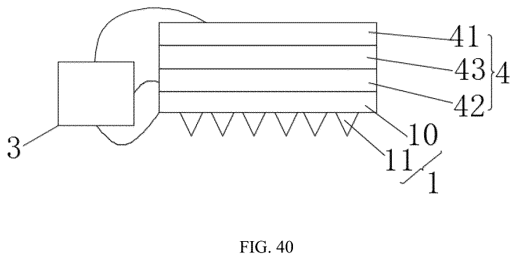

This technical solution is a further modification based on technical solution 2. As shown in FIG. 40, disclosed is a closed-loop control system according to an embodiment of the disclosure. The closed-loop control system includes an electroosmotic pump 4, a diabetes sensor 1, and a signal conversion module 3.

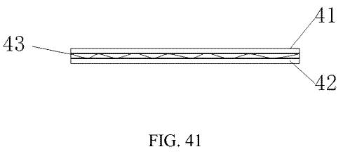

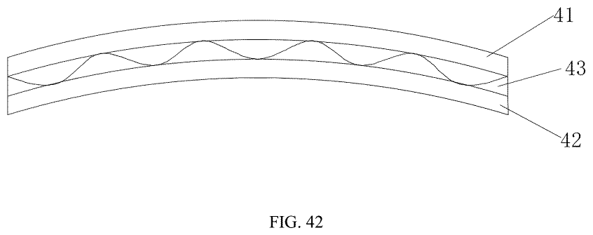

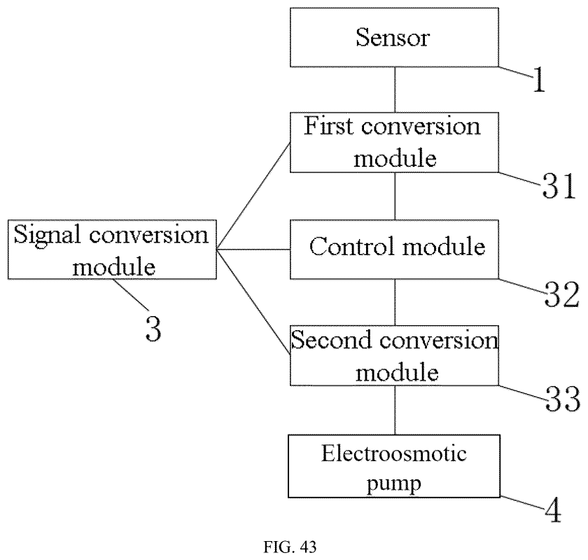

In some embodiments, as shown in FIG. 40, FIG. 41, FIG. 42 and FIG. 43, the electroosmotic pump 4 includes a first electrode layer 41, a second electrode layer 42, and an intermediate film layer 43, wherein the intermediate film layer 43 is located between the first electrode layer 41 and the second electrode layer 42.

The intermediate film layer 43 has a plurality of perforations (not shown). In some embodiments, the intermediate film layer 43 is made of a flexible film material, such as a polycarbonate film with perforations, a polyester film with perforations, a polytetrafluoroethylene film with perforations, or a polyimide film with perforations, which are not specifically limited here. In some embodiments, the intermediate film 43 is made of a hard film material.

In some embodiments, the first electrode layer 41 and the second electrode layer 42 are made of a hard film material, such as 304 stainless steel mesh, 316 stainless steel mesh; a metal mesh, such as an aluminum mesh, a titanium mesh, a platinum mesh; a metal coating, such as a gold metal coating, a platinum metal coating; or a gold-plated stainless steel mesh. In some embodiments, the first electrode layer 41 and the second electrode layer 42 are made of a flexible film material.

As shown in FIG. 42, in some embodiments, when the first electrode layer 41, the second electrode layer 42, and the intermediate film layer 43 are all made of flexible film materials, the electroosmotic pump could be bent integrally, which is more conducive to the use of the electroosmotic pump in various environments.

When the electroosmotic pump 4 is turned on, that is to say, the first electrode layer 41 and the second electrode layer 42 are powered on, the first electrode layer 41 and the second electrode layer 42 apply an electric field to the intermediate film layer 43, such that an electrical double layer is formed on the inner wall of perforations on the intermediate film layer 43. Under the action of the electric field, the electric charge in the electrical double layer moves in the direction of the electrode with opposite charge, and drags the surrounding liquid to flow, thereby providing continuous infusion.

As shown in FIG. 40, in some embodiments, the substrate 10 is connected with the second electrode layer 42, and the tip end of the microneedle array 11 faces the side away from the second electrode layer 42.

Specifically, when the closed-loop control system is used, the insulin solution is located at the position of the first electrode layer 41. After the electroosmotic pump 4 is powered on, the insulin flows to the second electrode layer 42 under the driving effect of the intermediate film layer 43, and flows to the substrate 10 of the diabetes sensor 1 through the second electrode layer 42. Finally, the insulin solution is injected into the patient through the hollow microneedles 111 on the substrate 10, so as to achieve insulin injection.

Further, the input end of the control module 32 is connected with the output end of the diabetes sensor 1, and the output end thereof is connected with the input end of the electroosmotic pump 4. Therefore, the control module 32 could receive the electrical signal output by the diabetes sensor 1. Since the microneedles 111 on the diabetes sensor 1 enter the patient's body and contact the patient's subcutaneous interstitial fluid, it therefore could measure the glucose concentration in the patient's subcutaneous interstitial fluid. Further, the glucose concentration in the interstitial fluid is highly correlated with the blood glucose concentration, and thus the electrical signal output by the diabetes sensor 1 could reflect the blood glucose concentration. For example, the diabetes sensor 1 could detect a current at a constant voltage, and the intensity of the current signal is directly proportional to the glucose concentration.

The control module 32 could control the opening or closing of the electroosmotic pump 4 (i.e., powering on or off the electroosmotic pump 4) according to the electrical signal. For example, a preset value could be set in the control module 32. If the value of the electrical signal is not less than the preset value, the electroosmotic pump 4 is powered on. If the value of the electrical signal is less than the preset value, the electroosmotic pump 4 would not be powered on.

In this way, the electroosmotic pump 4 could be controlled according to the real-time blood glucose concentration of the patient.

When using the closed-loop control system with the electroosmotic pump, one end of the diabetes sensor 1 enters the patient's body and contacts the patient's subcutaneous interstitial fluid to measure the glucose concentration in the patient's subcutaneous interstitial fluid. Because the glucose concentration in the interstitial fluid is highly correlated with the blood glucose concentration, the signal output by the diabetes sensor 1 could reflect the blood glucose concentration;

Specifically, the diabetes sensor 1 could detect a current at a constant voltage, and the intensity of current signal is directly proportional to the glucose concentration. In addition to detecting the current signal, the first conversion module 31 also provides a constant voltage for the diabetes sensor 1. In some embodiments, the constant voltage is 0.1 V, −0.1 V, or 0.6 V, or other different voltages.

In some embodiments, the diabetes sensor 1 is located outside the patient's body, the signal conversion module 3 and the electroosmotic pump 4 are sequentially provided at one end of the diabetes sensor 1. The electroosmotic pump 4 is provided in close proximity to the skin of the patient to realize the injection of insulin for the patient. In addition, the diabetes sensor 1 with a microneedle array could penetrate deep into the dermis or fat layer of the patient, which is more effective for injecting insulin into the fat layer.

Specifically, the second conversion module 33 could provide a constant voltage to drive the electroosmotic pump and further control the injection dosage of insulin by controlling the voltage level and duration. In some embodiments, the voltage ranges from 0.1 V to 20 V.

In this way, after measuring the glucose concentration and generating an electrical signal by the diabetes sensor 1, the first conversion module 31 of the signal conversion module 3 receives and converts the electrical signal, and then sends the converted electrical signal to the control module 32. After receiving the electrical signal converted by the first conversion module 31, the control module 32 generates different instruction information according to the different electrical signals. For example, the control module 32 could generate an opening instruction or a closing instruction, and sends the generated instruction to the second conversion module 33, which then converts the received instruction into the corresponding signal and controls the opening or closing of the electroosmotic pump 4 according to the signal, such that the electroosmotic pump 4 could be controlled according to the real-time blood glucose concentration of the patient.

In some feasible embodiments, the first conversion module 31 is a first signal converter, the control module 32 is a microcontroller, and the second conversion module 33 is a second signal converter.

Specifically, those skilled in the art could utilize devices in the related technology as the first signal converter and the second signal converter, as long as the opening or closing of the electroosmotic pump 4 could be controlled by the control module 32. Therefore, there is no particular limitation on them, and the specific description of the related technology will not be repeated.

Technical Solution 4: Closed-Loop Control System with Electrochemical Pump

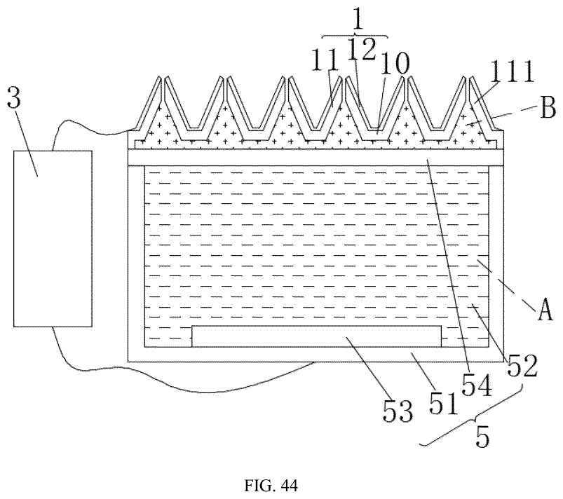

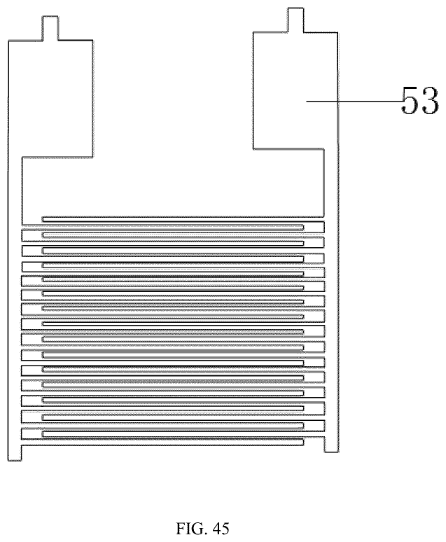

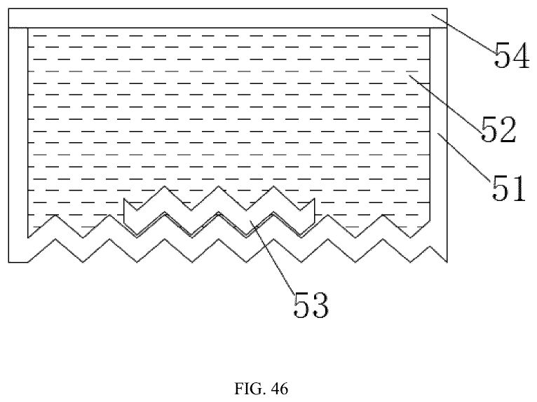

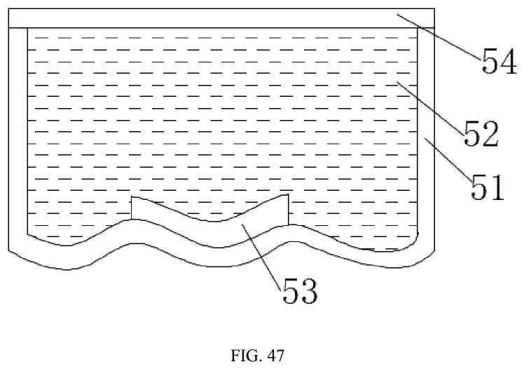

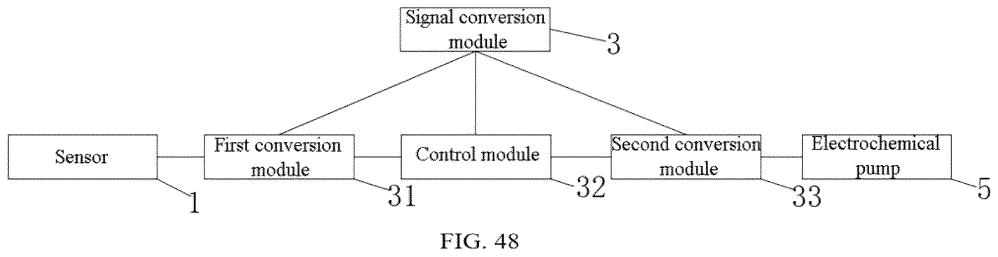

This technical solution is a further modification based on Technical solution 2. As shown in FIG. 44, FIG. 45, FIG. 46, FIG. 47 and FIG. 48, disclosed is a closed-loop control system according to the disclosure. The closed-loop control system includes an electrochemical pump 5, a diabetes sensor 1, and a signal conversion module 3.

In some embodiments, the electrochemical pump 5 includes a pump body 51, which has an accommodation zone A, in which an electrolyte solution 52 and an electrode layer 53 are arranged, wherein the electrode layer 53 is on the inner wall of the pump body 51, and an expanded film 54 covering the accommodation zone A is arranged on the pump body 51. In some embodiments, the pump body 51 is a cylindrical or hemispherical shape as a whole, the electrode layer 53 is an interdigital electrode made of platinum, the medium solution 52 is deionized water or a salt solution, and the expanded film 54 is a polytetrafluoroethylene film.

As shown in FIG. 45, in some embodiments, the interdigital electrodes 13 include alternating platinum finger electrodes, and the platinum finger electrodes each have a width of 1-500 μm. The interdigital electrodes 13 are connected to the outside of the pump body 51 through a conductive wire to receive current. In some embodiments, the electrode layer 53 is made of gold, silver, aluminum, carbon, etc. In some embodiments, the electrode layer 53 has an area of 1 mm²-1 cm², and the electrode layer 53 has a thickness of 50 nm-100 μm. In some embodiments, when the electrode layer 53 is an electrode of other shapes, such as a planar electrode, the planar electrode has a width of millimeters to centimeters. In some embodiments, the electrode layer 53 is formed on the substrate by sputtering or evaporation deposition of micro/nanofabrication, or by screen printing.

In some embodiments, the inner wall of the pump body 51 where the electrode layer 53 is located serves as the substrate of the electrode layer 53, wherein the substrate is in a shape of plane, sawtooth or curved surface. In some embodiments, the substrate is made of a flexible material, such as polyethylene terephthalate (PET), polyimide, parylene, polyurethane, polycarbonate, polyester, thermoplastic polyurethane elastomer (TPU), polyvinyl chloride (PVC), chitosan, polylactic acid, silica gel, rubber, latex, thermoplastic elastomer (TPE), perfluoroethylene propylene copolymer (FEP) and polytetrafluoroethylene (PTFE); and the substrate is made of a hard material such as glass.

In some embodiments, polydimethylsiloxane (PDMS), polyacrylate, silica gel (e.g., Ecoflex™, Dragon Skin™), rubber (e.g., NBR, IIR), latex, polyurethane, parylene, polyimide, and other materials is selected as a material for the expanded film 54.

In some embodiments, the substrate 10 and the microneedle array 11 are integrally formed, that is to say, the substrate 10 and the microneedle array 11 are fabricated by the same method or the same step. The microneedle array 11 includes a plurality of microneedles 111. In some embodiments, the microneedles 111 are a cone or pyramid with a certain height, and the interior of the microneedles 111 is hollow, with two penetrated ends, thereby forming an injection channel B, such that the insulin solution could be injected into the patient through the injection channel B of the microneedles 111.

In some embodiments, the expanded film 54 is connected with the substrate 10 of the diabetes sensor 1, and the tip end of the microneedle array 11 faces the side away from the expanded film 54.

Specifically, after the electrochemical pump 5 is powered on, the electrode layer 53 electrolyzes water, and hydrogen bubbles and oxygen bubbles are generated. These bubbles move towards the position where the expanded film 54 is located, and under the action of these bubbles, the expanded film 54 deforms and expands, and squeezes the insulin solution inside the hollow microneedle, such that the insulin solution flows out through the injection hole. When the diabetes sensor 1 is applied to the patient, the insulin solution could be injected into the patient. When the electrochemical pump 5 is not powered on, hydrogen and oxygen are recombined into water in the catalysis of the electrode layer 53, and then the expanded film 54 shrinks, such that the insulin solution would no longer flow out of the injection hole at the tip of the microneedles 111.

Further, an input end of the control module 32 is connected with an output end of the diabetes sensor 1, and an output end thereof is connected with an input end of the electrochemical pump 5. Therefore, the control module 32 could receive the electrical signal output by the diabetes sensor 1. Since the microneedles 111 on the diabetes sensor 1 enter the patient's body and contact the patient's subcutaneous interstitial fluid, it could measure the glucose concentration in the patient's subcutaneous interstitial fluid. Further, the glucose concentration in interstitial fluid is highly correlated with the blood glucose concentration, such that the electrical signal output by the diabetes sensor 1 could reflect the blood glucose concentration. For example, the diabetes sensor 1 could detect a current at a constant voltage, and the intensity of the current signal is directly proportional to the level of the glucose concentration.

The control module 32 could control opening or closing of the electrochemical pump 5 (i.e., powering on or off the electrochemical pump 5) according to the electrical signal. For example, in some embodiments, a preset value is set in the control module 32. If the value of the electrical signal is not less than the preset value, the electrochemical pump 5 would be powered on. If the value of the electrical signal is less than the preset value, the electrochemical pump 5 would not be powered on.

In this way, the electrochemical pump 5 could be controlled according to the real-time blood glucose concentration of the patient.

As shown in FIG. 48, in some embodiments, the signal conversion module 3 includes a first conversion module 31, a control module 32, and a second conversion module 33. Specifically, an input end of the first conversion module 31 is connected with an output end of the diabetes sensor 1, an output end of the first conversion module 31 is connected with an input end of the control module 32, an input end of the second conversion module 33 is connected with an output end of the control module 32, and an output end of the second conversion module 33 is connected with an input end of the electrochemical pump 5.

When using the closed-loop control system with the electrochemical pump, one end of the diabetes sensor 1 enters the patient's body and contacts the patient's subcutaneous interstitial fluid to measure the glucose concentration in the patient's subcutaneous interstitial fluid. Because the glucose concentration of the interstitial fluid is highly correlated with the blood glucose concentration, the signal output by the diabetes sensor 1 could reflect the blood glucose concentration. In some embodiments, the diabetes sensor 1 is located outside the patient's body, and the signal conversion module 3 and the electrochemical pump 5 are sequentially provided at one end of the diabetes sensor 1. The electrochemical pump 5 is provided in close proximity to the skin of the patient to realize the injection of insulin for the patient. In addition, the diabetes sensor 1 with tubular substrate could enter the dermis or fat layer of the patient, which is more effective for injecting insulin into the fat layer.

Specifically, the second conversion module 33 could provide a constant voltage to drive the electrochemical pump and further control the injection dosage of insulin by controlling the voltage level and duration. In some embodiments, the voltage ranges from 0.1 V to 20 V.

In this way, after measuring the glucose concentration and generating an electrical signal by the diabetes sensor 1, the first conversion module 31 of the signal conversion module 3 receives and converts the electrical signal, and then sends the converted electrical signal to the control module 32. After receiving the electrical signal converted by the first conversion module 31, the control module 32 generates different instruction information according to different electrical signals. For example, the control module 32 could generate an opening instruction or a closing instruction. Also, the control module 32 sends the generated instruction to the second conversion module 33, which converts the received instruction into the corresponding signal, and controls opening or closing of the electrochemical pump 5 according to the signal, such that the electrochemical pump 5 could be controlled according to the real-time blood glucose concentration of the patient.

In some feasible embodiments, the first conversion module 31 is a first signal converter, the control module 32 is a microcontroller, and the second conversion module 33 is a second signal converter.

Specifically, those skilled in the art could utilize devices in related technology as the first signal converter and the second signal converter, as long as the effect of controlling opening or closing of the electrochemical pump 5 could be achieved by the control module 32. Therefore, there is no particular limitation on them, and the specific description of the related technology will not be repeated.

In the description of the present disclosure, it should be noted that the orientation or positional relationship indicated by the terms “center”, “top”, “bottom”, “left”, “right”, “vertical”, “horizontal”, “inside/inner”, “outside” and so on is based on the orientation or positional relationship shown in the accompanying drawings, only for the convenience of describing the present disclosure and simplifying the description, rather than indicating or implying that the device or element referred to must have a specific orientation, be constructed and operated in a specific orientation, and therefore it cannot be understood as a limitation to the present disclosure. In addition, the terms “first” and “second” are only used for descriptive purposes and cannot be understood as indicating or implying relative importance.

Specific embodiments are described in the present disclosure to illustrate the principle and embodiment mode of the disclosure. The description of the above embodiments is only used to help understand the method and core idea of the disclosure. Further, according to the idea of the disclosure, changes could be made based on the specific embodiment modes and scope for ordinary technicians in the art. To sum up, the content of the specification should not be construed as a limitation to the disclosure.

What is claimed is:

1. A diabetes sensor, comprising
a substrate,
a microneedle array arranged on one side of the substrate, and
a plurality of electrodes covering the microneedle array and the substrate,
wherein the microneedle array comprises a plurality of microneedles; and
wherein the plurality of electrodes comprises an electrochemical sensor and a reverse iontophoresis device,
the electrochemical sensor being configured to detect glucose molecules in interstitial fluid and generate an electric signal, and
the reverse iontophoresis device being configured to generate a reverse iontophoresis effect to attract the glucose molecules from a deep skin layer to an upper part of dermis where needle tips of the microneedles are located.

2. The diabetes sensor as claimed in claim 1, wherein the microneedles each have a height of not less than 100 μm and not more than 1000 μm.

3. The diabetes sensor as claimed in claim 1, wherein the electrochemical sensor comprise a working electrode and a counter electrode, or comprises a working electrode, a reference electrode, and a counter electrode; and
the reverse iontophoresis device comprises a positive electrode and a negative electrode;
wherein the working electrode of the electrochemical sensor and the negative electrode of the reverse iontophoresis device form interdigital electrodes;
glucose oxidase is immobilized on the working electrode of the electrochemical sensor; and
the counter electrode of the electrochemical sensor and the positive electrode of the reverse iontophoresis device are located on one side or two sides of the interdigital electrodes.

4. The diabetes sensor as claimed in claim 1, wherein the microneedle array comprises a solid microneedle array or a hollow microneedle array.

5. The diabetes sensor as claimed in claim 1, wherein the substrate and the microneedle array are each independently made of a material comprising one selected from the group consisting of a polymeric material, a biodegradable material, and a biocompatible material.

6. The diabetes sensor as claimed in claim 3, wherein the working electrode of the electrochemical sensor is made of a material comprising one selected from the group consisting of gold, platinum, carbon, a gold composite, a platinum composite, a carbon composite, and silver/silver chloride;
the counter electrode of the electrochemical sensor is made of a material comprising one selected from the group consisting of gold, platinum, carbon, a gold composite, a platinum composite, a carbon composite, and silver/silver chloride; and
the reverse iontophoresis device is made of a material comprising one selected from the group consisting of silver/silver chloride, a silicone material, a conductive polymer, graphene, and gold.

7. A method for manufacturing a diabetes sensor, being applicable to the diabetes sensor as claimed in claim 1, and comprising the steps of
providing a substrate;
forming a microneedle array on one side of the substrate, wherein the microneedle array comprises a plurality of microneedles; and
forming a plurality of electrodes on the substrate and the microneedle array,
wherein the plurality of electrodes comprises an electrochemical sensor and a reverse iontophoresis device,
the electrochemical sensor being configured to detect glucose molecules in interstitial fluid and generate an electric signal; and
the reverse iontophoresis device being configured to generate a reverse iontophoresis effect to attract the glucose molecules from a deep skin layer to an upper part of dermis where needle tips of the microneedles are located.

8. The method as claimed in claim 7, wherein forming the microneedle array on one side of the substrate comprises
providing a mold with a microneedle sequence that matches the microneedle array; and
pouring a polymeric material into the mold, solidifying the polymeric material, and then peeling a resulting solidified polymeric material off the mold, to obtain the microneedle array.

9. The method as claimed in claim 7, wherein forming the microneedle array on one side of the substrate comprises
forming the microneedle array on the substrate by a 3D printing process or a micro/nanofabrication process.

10. The method as claimed in claim 7, wherein forming the plurality of electrodes on the substrate and the microneedle array comprises
forming the plurality of electrodes by a micro/nanofabrication process, a screen printing process, or an aerosol jet printing process.

11. A closed-loop control system, comprising
a pump,
a signal conversion module, and
the diabetes sensor as claimed in claim 1,
wherein one end of the pump is connected with the substrate of the diabetes sensor, and a tip end of the microneedle array faces a side away from the pump; and
the signal conversion module comprises a first conversion module, a control module, and a second conversion module,
wherein an input end of the first conversion module is connected with an output end of the diabetes sensor, and an output end of the first conversion module is connected with an input end of the control module, and the first conversion module is configured to receive and convert an electrical signal output by the diabetes sensor;
the control module is configured to receive the electrical signal converted by the first conversion module and send an instruction to the second conversion module according to the electrical signal received; and
an input end of the second conversion module is connected with the output end of the control module, and an output end of the second conversion module is connected with an input end of the pump; and the second conversion module is configured to receive and convert the instruction output by the control module, and send converted instruction signal to the pump to control opening or closing of the pump.

12. The closed-loop control system as claimed in claim 11, wherein the first conversion module is a first signal converter;
the control module is a microcontroller; and
the second conversion module is a second signal converter.

13. The closed-loop control system as claimed in claim 11, wherein the pump is an ultrasonic pump, the ultrasonic pump comprising an upper casing, a lower casing, and a thin film arranged between the upper casing and the lower casing,
wherein a drug storage chamber is formed between the thin film and the upper casing, a plurality of conical holes is distributed on the thin film, and a large-diameter end of each of the conical holes is adjacent to the drug storage chamber;
a piezoelectric circular ring is arranged on one side of the thin film facing away from the drug storage chamber;
the lower casing is provided with a liquid outlet, the liquid outlet being connected with the substrate of the diabetes sensor; and
the tip end of the microneedle array faces a side away from the liquid outlet.

14. The closed-loop control system as claimed in claim 13, wherein the thin film is made of a material comprising at least one selected from the group consisting of stainless steel, gold, copper, zinc, platinum, silver, tungsten, aluminum, an aluminum alloy, natural rubber, isoprene rubber, polybutadiene rubber, styrene butadiene rubber, nitrile rubber, chloroprene rubber, butyl rubber, halogenated butyl rubber, ethylene propylene rubber, ethylene propylene diene rubber, epichlorohydrin rubber, polyacrylate rubber, silicone rubber, fluorosilicone rubber, fluororubber, chlorosulfonated polyethylene, hydrogenated nitrile rubber, thermoplastic polyolefin elastomer, thermoplastic styrene elastomer, polyurethane thermoplastic elastomer, polyester thermoplastic elastomer, polyamide thermoplastic elastomer, halogen containing thermoplastic elastomer, ionic thermoplastic elastomer, ethylene copolymer thermoplastic elastomer, 1,2-poly-butadiene thermoplastic elastomer, trans polyisoprene thermoplastic elastomer, melt processible thermoplastic elastomer, thermoplastic vulcanizate, and polydimethylsiloxane; and
the piezoelectric ring is made of a piezoelectric crystal, a piezoelectric ceramic, and a piezoelectric polymer.

15. The closed-loop control system as claimed in claim 11, wherein the pump is an electroosmotic pump, the electroosmotic pump comprising a first electrode layer, a second electrode layer, and an intermediate film layer,
wherein the intermediate film layer is located between the first electrode layer and the second electrode layer, and a plurality of perforations are distributed on the intermediate film layer; and
the substrate is connected with the second electrode layer, and the tip end of the microneedle array faces a side away from the second electrode layer.

16. The closed-loop control system as claimed in claim 15, wherein the first electrode layer, the second electrode layer, and the intermediate film layer are each independently made of a material comprising a hard film material or a flexible film material.

17. The closed-loop control system as claimed in claim 11, wherein the pump is an electrochemical pump, the electrochemical pump comprising a pump body,
wherein the pump body has an accommodation zone accommodating an electrolyte solution and an electrode layer connected to an inner wall of the pump body;
the pump body is provided with an expanded film covering the accommodation zone;
the expanded film is connected with the substrate of the diabetes sensor, and the tip end of the microneedle array faces a side away from the expanded film.

18. The closed-loop control system as claimed in claim 17, wherein the expanded film is made of a material comprising at least one selected from the group consisting of polytetrafluoroethylene, polydimethylsiloxane, polyacrylate, silicone, rubber, latex, polyurethane, parylene, and polyimide; and
the electrode layer is made of a material comprising a hard film material or a flexible film material.

19. The closed-loop control system as claimed in claim 11, wherein the microneedles each have a height of not less than 100 μm and not more than 1000 μm.

20. The closed-loop control system as claimed in claim 11, wherein the microneedle array comprises a solid microneedle array or a hollow microneedle array.
